(19) United States
(12) Patent Application Publication
(10) Pub. No.: US 2026/0157932 A1
(43) Pub. Date: Jun. 11, 2026

(54) DEVICES AND METHODS FOR DELIVERY OF MULTI-COMPONENT FLUIDS

(71) Applicant: TYBR Health, Inc., Houston, TX (US)

(72) Inventors: Peter Alexander SMITH, Houston, TX (US); Timothy J. KEANE, Jr., Houston, TX (US)

(21) Appl. No.: 19/181,151

(22) Filed: Apr. 16, 2025

Related U.S. Application Data
(60) Provisional application No. 63/635,366, filed on Apr. 17, 2024.

(51) Int. Cl. A61J 1/20; A61K 35/12; A61M 31/00; A61P 41/00

(52) U.S. Cl. CPC A61J 1/2093; A61K 35/12; A61M 31/00; A61P 41/00; A61M 2205/586; A61M 2206/20

(57) ABSTRACT

Provided herein are devices and methods for mixing and delivering a multi-component fluid capable of decreasing post-surgical adhesions. The devices are configured to impart shear stress on the multi-component fluid and increase yield stress of a resulting gel. In some cases, the devices can be airless. In some cases, the devices can use gases to impart additional shear stress on the fluids in the devices. Mixers for imparting sheet stress can include mixer stages, vortex inducers, spacers, or any combination thereof.

### CROSS-REFERENCE TO RELATED APPLICATIONS

This application claims the benefit of U.S. provisional application No. 63/635,366, filed Apr. 17, 2024, the complete disclosure of which is incorporated herein by reference for all purposes.

### TECHNICAL FIELD

Devices and methods for delivery of multi-component fluids are described. In particular, delivery devices for a multi-component fluid that can prevent tissue adhesion, and more specifically, tissue adhesion post-surgery, along with methods of using these delivery devices, are described.

### BACKGROUND

Surgical procedures can sometimes result in negative outcomes. Tissue adhesions are considered a frequent complication of abdominal surgery. Such adhesions may cause acute abdominal bowel obstruction, infertility, loss of range of motion, or chronic pain, and patients may require reoperation or other medical intervention to treat resulting comorbidities. Adhesions on the bowel are currently the number one cause of small bowel obstruction in the United States resulting in about 400,000 emergency surgeries for intestinal obstruction repair procedures, with an estimated 300,000 of the 400,000 resulting from post-surgical adhesions.

### SUMMARY

Provided herein is a device for delivery of a multi-component fluid. In some embodiments, the device comprises a housing. The housing can have a first lumen and a second lumen. In some embodiments, the first lumen is configured to receive a first component of the multi-component fluid. In some embodiments, the second lumen is configured to receive a second component of the multi-component fluid. In some cases, a mixer is coupled to the housing. The mixer can comprise one or more stages. The mixer can comprise one or more vortex inducers. In some embodiments, a proximal end of the mixer is in fluid communication with the first lumen and with the second lumen. In some embodiments, the proximal end is configured to receive the first component and the second component within the mixer and to mix the first component and the second component to form the multi-component fluid. In some embodiments, a nozzle is coupled to the mixer. The nozzle can comprise a nozzle outlet. In some embodiments, the nozzle is configured to receive the multi-component fluid from the mixer and deliver the multi-component fluid from the nozzle outlet.

In some embodiments, the nozzle further comprises one or more spacers. In some embodiments, at least one vortex inducer of the one or more vortex inducers is located distal to the one or more spacers and proximal to the mixer. In some embodiments, one or more vortex inducers are located proximal to the one or more spacers. In some embodiments, the one or more spacers are proximal to at least one vortex inducer of the one or more vortex inducers, and the one or more spacers are distal to at least a second vortex inducer. In some embodiments, a distal end of the spacer is narrower than a proximal end of the spacer. In some embodiments, the nozzle comprises two vortex inducers. In some embodiments, a vortex inducer of the one or more vortex inducers comprises the nozzle outlet. In some embodiments, the one or more vortex inducers comprise high shear mixers of the multi-component fluid. In some cases, the mixer comprises 12 mixer stages. In some cases, the 12 mixer stages are configured to form an array of mixer stages. In some cases, the array of mixer stages further comprises the one or more vortex inducers, or the one or more spacers, or both. In some embodiments, the mixer is a high shear mixer. In some embodiments, the vortex inducer is configured to induce a rotational component in the multi-component fluid flowing therethrough. In some embodiments, the vortex inducer imparts shear stress on the multi-component fluid flowing therethrough. In some embodiments, the shear stress imparted on the multi-component can result in a gel comprising a yield stress of at least 200 Pa. In some embodiments, the shear stress imparted on the multi-component can result in a gel comprising a yield stress of between about 200 Pa to about 700 Pa. In some embodiments, the mixer comprises a baffle, a blade, a channel, a semi-circular cylinder, a slot, a plate, a fin, or a combination thereof.

In some cases, the first component or the second component comprises a buffer. In some cases, the buffer solution is a phosphate buffered saline. In some cases, the multi-component fluid is buffered to a mildly acidic pH. In some cases, the multi-component fluid is buffered to a pH from about 6.5 to about 8.0. In some cases, the first component or the second component comprises an extracellular (ECM) matrix material. In some embodiments, the multi-component fluid comprises a hydrogel.

Described herein is a method of delivering a multi-component fluid for treatment of a wound. In some cases, the method comprises delivering a first component to a first lumen and a second component to a second lumen of a delivery device. In some cases, the delivery device comprises a housing. The housing can have the first lumen and the second lumen. The housing can have an actuator. In some embodiments, a distal end of the actuator is coupled to a proximal end of the housing. In some embodiments, the actuator is configured to activate movement of the first component through the first lumen to the mixer, the second component through the second lumen to the mixer, or both. In some embodiments, a mixer can be coupled to the housing. In some embodiments, the mixer comprises one or more mixer stages and one or more vortex inducers. In some embodiments, a proximal end of the mixer is in fluid communication with the first lumen and with the second lumen. In some embodiments, the proximal end is configured to receive the first component and the second component within the mixer and to mix the first component and the second component to form the multi-component fluid. In some cases, a nozzle can be coupled to the mixer. In some embodiments, the nozzle comprises a nozzle outlet. In some embodiments, the nozzle is configured to receive the multi-component fluid from the mixer and deliver the multi-component fluid from the nozzle outlet. In some cases, the method comprises activating the actuator so that the mixer receives the first component and the second component. The method can comprise further activating the actuator so that the first component and the second component mix to form the multi-component fluid. In some cases, the method comprises further activating the actuator so that the multi-component fluid is delivered from the nozzle outlet onto a patient's wound.

In some cases, the first lumen of the delivery device is in fluid communication with a first container, such that the first lumen is configured to receive the first component of the multi-component fluid from the first container. In some cases, the second lumen of the delivery device is in fluid communication with a second container, such that the second lumen is configured to receive the second component of the multi-component fluid from the second container. In some cases, the actuator of the delivery device comprises a piston, a plunger, a pump, or a multi-stage button. In some cases, the delivery device can comprise any of the devices described above.

Provided herein is a method for delivering a multi-component fluid. In some cases, the method comprises delivering a first component and a second component of the multi-component fluid through a first lumen and a second lumen, respectively, of a delivery device. The delivery device can comprise a housing. In some cases, the housing has the first lumen and the second lumen. The delivery device can comprise a mixer coupled to the housing. In some cases, the mixer comprises one or more mixer stages and one or more vortex inducers. In some cases, a proximal end of the mixer is in fluid communication with the first lumen and with the second lumen. In some cases, the proximal end is configured to receive the first component and the second component within the mixer and to mix the first component and the second component to form the multi-component fluid. In some embodiments, the delivery device comprises a nozzle coupled to the mixer. In some cases, the nozzle comprises a nozzle outlet. In some cases, the nozzle is configured to receive the multi-component fluid from the mixer and deliver the multi-component fluid from the nozzle outlet. In some embodiments, the method can comprise delivering the multi-component fluid through one or more vortex inducers in the mixer. In some embodiments, the method can comprise delivering the multi-component fluid through the nozzle outlet. In some cases, the delivery device comprises any of the devices described above.

Described herein is a method of mixing two fluids to create a multi-component fluid. In some embodiments, the method can comprise delivering a first component and a second component of the multi-component fluid through a first lumen and a second lumen, respectively, of a delivery device. In some embodiments, the delivery device comprises a housing and a mixer. In some embodiments, the housing comprises the first lumen and the second lumen. In some embodiments, the mixer is coupled to the housing. In some embodiments, the mixer comprises one or more mixer stages and one or more vortex inducers. In some embodiments, a proximal end of the mixer is in fluid communication with the first lumen and with the second lumen. In some embodiments, the proximal end is configured to receive the first component and the second component within the mixer and to mix the first component and the second component to form the multi-component fluid. In some embodiments, the method comprises delivering the first component and the second component to the mixer. In some embodiments, delivering comprises moving the first component and the second component from the first lumen and the second lumen, respectively, to a third lumen comprising both the first component and the second component. In some embodiments, the method comprises imparting shear stress onto the multi-component fluid in the third lumen. In some cases, the delivery device comprises any of the devices described above.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIGS. 1A and 1B illustrate an exemplary embodiment of a fluid delivery device as described herein, in which:

FIG. 1A shows a partial transparent perspective view of the fluid delivery device; and

FIG. 1B shows a partial transparent perspective exploded view of the fluid delivery device of FIG. 1A.

FIG. 2 illustrates a partial transparent perspective exploded view of another exemplary embodiment of a fluid delivery device as described herein.

FIG. 3 depicts a partial transparent perspective view of still another exemplary embodiment of a fluid delivery device as described herein.

FIG. 4A illustrates a side perspective view of yet another exemplary embodiment of a fluid delivery device as described herein.

FIG. 4B illustrates a perspective top view of even still another exemplary embodiment of a fluid delivery device as described herein.

FIG. 5A depicts a side section view of an exemplary embodiment of a mixing element shown in use with a fluid delivery device as described herein.

FIG. 5B depicts an exploded side perspective section view of an exemplary embodiment of a mixing element shown with a fluid delivery device as described herein.

FIG. 6 shows an expanded perspective view of an exemplary embodiment of mixers of the mixing element referred to in FIGS. 5A and 5B.

FIG. 7 shows an expanded perspective view of an exemplary embodiment of a spacer of the mixing element referred to in FIGS. 5A and 5B.

FIGS. 8A and 8B depict perspective proximate and perspective distal views, respectively, of a vortex inducing component of the mixing element referred to in FIGS. 5A and 5B.

FIG. 9A illustrates a graph of yield stress of a multi-component hydrogel when embodiments of devices described herein are used on tissue.

FIG. 9B illustrates a graph of yield stress of a multi-component hydrogel when embodiments of fluid delivery devices described herein are used.

### DETAILED DESCRIPTION

Post-surgical tissue adhesions can form between organs or tissues after surgery, connecting them abnormally. These adhesions develop as the body attempts to repair itself and is a normal response that can occur after surgery, infection, injury, trauma or radiation. The tissue adhesions can appear as thin sheets of tissue, or thick fibrous, scar-like bands, and present a significant complication to a patient after surgery, including pain, bowel obstruction, and even infertility. Adhesions may cause prolonged operative time in subsequent surgeries, increased dosages of anesthesia to complete the prolonged surgery, and an increased risk of complications such as hemorrhaging.

There are currently few reliable methods or devices for preventing the formation of post-surgical adhesions. In response to this unmet need, one or more embodiments of the present disclosure provides for devices, systems, and methods for preventing the formation of post-surgical adhesions in a patient.

Disclosed herein are devices and methods for mixing and delivering a multi-component fluid capable of decreasing post-surgical adhesions, such as by forming a tissue barrier to adhesion. Each component of the multi-component fluid may be contained in a separate container. The method may comprise delivering a component of the multi-component fluid from a container to a multi-lumen tube (e.g., by using a plunging system), where the components of the multi-component fluid may be kept separate from one another. The components of the multi-component fluid may be subjected to mixing in a mixer. The multi-component fluid may be ejected from a nozzle positioned distal to a mixer.

Provided herein is a device for delivery of a multi-component fluid. The devices can be configured to impart shear stress on the multi-component fluid and increase yield stress of the resulting gel. The device can comprise a housing comprising a distal end and a proximal end. The housing can have a first lumen and a second lumen, each lumen extending from the proximal end to the distal end of the housing. The first lumen can be configured to receive a first component of the multi-component fluid. The second lumen can be configured to receive a second component of the multi-component fluid. In some cases, a mixer is coupled to the distal end of the housing. The mixer can comprise one or more stages. A proximal end of the mixer can be in fluid communication with the first lumen and with the second lumen. The proximal end can be configured to receive the first component and the second component within the mixer and to mix the first component and the second component to form the multi-component fluid. The device can comprise a nozzle disposed distal to the mixer. The nozzle can comprise one or more vortex inducers, one or more spacers, and a nozzle outlet. The nozzle can receive the multi-component fluid from the mixer and deliver the multi-component fluid from the nozzle outlet.

### Multi-Component Fluid

In some embodiments, the multi-component fluid comprises two or more components (e.g., two or more fluids) that are initially liquid, but are configured to turn into a gel or begin to gel upon or after being mixed together. In some embodiments, multi-component fluid may form or begin to form a gel after mixing and/or upon the application of heat. In some embodiments, the multi-component fluid may form or begin to form a gel within a time frame after mixing. In some embodiments, the multi-component fluid may form or begin to form a gel upon buffering to a neutral or a physiological pH (e.g., about 7). In some embodiments, the multi-component fluid may form or begin to form a gel upon mixing and upon buffering to a physiological pH (e.g., about 7). The multi-component fluid can be constituents of a hydrogel.

In some embodiments, the delivery device mixes the components of the multi-component fluid and delivers the multi-component fluid to a target (e.g., a target tissue) prior to the multi-component fluid being fully gelled. The time frame for the gel to form may be from about 1 second(s) to about 5 minutes (m). In some cases, it may be considered that a gel has formed when it has reached about 50% of its fully gelled stiffness. In some embodiments, the multi-component fluid gels in about 1 s to about 300 s after mixing. In some embodiments, the multi-component fluid gels in about 1 s to about 2 s after mixing, about 1 s to about 3 s after mixing, about 1 s to about 5 s after mixing, about 1 s to about 10 s after mixing, about 1 s to about 20 s after mixing, about 1 s to about 30 s after mixing, about 1 s to about 40 s after mixing, about 1 s to about 50 s after mixing, about 1 s to about 100 s after mixing, about 1 s to about 200 s after mixing, about 1 s to about 300 s after mixing, about 2 s to about 3 s after mixing, about 2 s to about 5 s after mixing, about 2 s to about 10 s after mixing, about 2 s to about 20 s after mixing, about 2 s to about 30 s after mixing, about 2 s to about 40 s after mixing, about 2 s to about 50 s after mixing, about 2 s to about 100 s after mixing, about 2 s to about 200 s after mixing, about 2 s to about 300 s after mixing, about 3 s to about 5 s after mixing, about 3 s to about 10 s after mixing, about 3 s to about 20 s after mixing, about 3 s to about 30 s after mixing, about 3 s to about 40 s after mixing, about 3 s to about 50 s after mixing, about 3 s to about 100 s after mixing, about 3 s to about 200 s after mixing, about 3 s to about 300 s after mixing, about 5 s to about 10 s after mixing, about 5 s to about 20 s after mixing, about 5 s to about 30 s after mixing, about 5 s to about 40 s after mixing, about 5 s to about 50 s after mixing, about 5 s to about 100 s after mixing, about 5 s to about 200 s after mixing, about 5 s to about 300 s after mixing, about 10 s to about 20 s after mixing, about 10 s to about 30 s after mixing, about 10 s to about 40 s after mixing, about 10 s to about 50 s after mixing, about 10 s to about 100 s after mixing, about 10 s to about 200 s after mixing, about 10 s to about 300 s after mixing, about 20 s to about 30 s after mixing, about 20 s to about 40 s after mixing, about 20 s to about 50 s after mixing, about 20 s to about 100 s after mixing, about 20 s to about 200 s after mixing, about 20 s to about 300 s after mixing, about 30 s to about 40 s after mixing, about 30 s to about 50 s after mixing, about 30 s to about 100 s after mixing, about 30 s to about 200 s after mixing, about 30 s to about 300 s after mixing, about 40 s to about 50 s after mixing, about 40 s to about 100 s after mixing, about 40 s to about 200 s after mixing, about 40 s to about 300 s after mixing, about 50 s to about 100 s after mixing, about 50 s to about 200 s after mixing, about 50 s to about 300 s after mixing, about 100 s to about 200 s after mixing, about 100 s to about 300 s after mixing, or about 200 s to about 300 s. In some embodiments, the multi-component fluid gels in about 1 s, about 2 s, about 3 s, about 5 s, about 10 s, about 20 s, about 30 s, about 40 s, about 50 s, about 100 s, about 200 s, or about 300 s after mixing. In some embodiments, the multi-component fluid gels in about 300 s to about 1000 s after mixing. In some embodiments, the multi-component fluid gels in about 1000 s after mixing. In some embodiments, the gel may reach a maximum strength (e.g., storage modulus) in between 5 minutes (min) to 30 min after gel initiates to form. In some embodiments, the multi-component fluid may be a shear thinning fluid and may experience a reduction in viscosity under shear strain (e.g., mixing). In some embodiments, the multi-component fluid comprises an acidic pre-gel which is buffered to a biological pH when it is mixed with a buffer solution and forms a gel with an increased storage modulus. In some embodiments, the multi-component fluid comprises an acidic ECM pre-gel which gels into a cross-linked ECM hydrogel scaffold when mixed and buffered to a neutral or physiological pH (e.g., about 7).

The multi-component fluid may comprise an adhesion barrier material. In some embodiments, the multi-component fluid may comprise a tissue-derived gel. In some embodiments, a first component comprising a tissue-derived stimuli-responsive gel with a viscosity ranging from about 5 centipoise (cP) to about 1,000,000 cP (1 cP=1 mPa*s) may be mixed with a second component comprising a buffer solution with a dynamic viscosity ranging from 0.5 cP to about 2.0 cP, to form a multi-component fluid. The multi-component fluid may form or begin to form a gel upon mixing. In some embodiments, the multi-component fluid (e.g., gel) has a viscosity similar to that of the tissue-derived stimuli-responsive gel. In some embodiments, a first component (e.g., first fluid) having a first viscosity range may be mixed with a second component (e.g., second fluid) having a second viscosity range, to form a multi-component fluid having a third viscosity range that encompasses the first and second viscosity ranges. In some embodiments, two or more fluids with viscosities similar to the gel and the buffer solution, described herein, may be mixed to form a multi-component fluid. In some embodiments, the multicomponent fluid has a viscosity of about 0.5 cP to about 1,000,000 cP. In some embodiments, generating a shear stress in the multi-component fluid (e.g., by mixing) changes a viscosity of the multi-component fluid. In some cases, the shear stress can be imposed on the fluid by a swirl or vortex inducer element in the mixer. In some cases, the shear stress can be increased by a swirl or vortex inducer element at the distal end of the nozzle.

In some embodiments, the multi-component fluid comprises a natural polymeric material, a polymeric material derived from a natural source, a synthetic polymeric material, or any combination thereof. In some embodiments, the natural polymeric material comprises collagen, gelatin, fibrin, alginate, agar, cassava, maize, chitosan, gellan gum, corn-starch, chitin, cellulose, chia (Salvia hispanica) recombinant silk, decellularized tissue (plant or animal), hyaluronic acid, glycosaminoglycans, fibronectin, laminin, hemicellulose, glucomannan, textured vegetable protein, heparan sulfate, chondroitin sulfate, tempeh, keratan sulfate, or any combination thereof. In some embodiments, the synthetic material comprises hydroxyapatite, polyethylene terephthalate, acrylates, polyethylene glycol, polyglycolic acid, polycaprolactone, polylactic acid, their copolymers, or any combination thereof. In some embodiments, the multi-component fluid comprises a hydrogel, such as alginate. In some embodiments, the multi-component fluid comprises cellulose, cellulose derivatives, gelatin, acrylic resins, glass, silica gels, polyvinyl pyrrolidine (PVP), co-polymers of vinyl and acrylamide, polyacrylamides, latex gels, dextran, cross-linked dextrans, rubber, silicon, plastics, nitrocellulose, natural sponges, metal, and agarose gel. In some embodiments, the multi-component fluid comprises a biomaterial such as silk, poly (ethylene glycol), agarose, polylactic acid, poly(acryl acmide), diacrylate, poly(vinyl acid), poly (lactic co-glycolic acid), poly(methyl methacrylate), lipids, metals, cellulose, chitin, chitosan, collagen, gelatin, fibrin, alginate, agar, cassava, maize, gellan gum, corn-starch, chia (Salvia hispanica), decellularized tissue (plant or animal), hyaluronic acid, fibronectin, laminin, hemicellulose, glucomannan, textured vegetable protein, heparan sulfate, chondroitin sulfate, keratan sulfate, pectin, lignin, Matrigel, or any combination thereof. In some embodiments, the multi-component fluid comprises a synthetic fluid, synthetic gel, buffer solution, natural fluid, or a natural gel such as a tissue-derived gel. A tissue derived gel may be autologous or allogenic in origin. A tissue derived gel may be blended with a synthetic gel or synthetic fluid. In some embodiments, the multi-component fluid comprises an extracellular matrix (ECM) gel. In some embodiments, a tissue derived gel comprises an extracellular matrix pre-gel and a pH buffer. The buffer may comprise a base (e.g., NaOH), a salt (e.g., PBS), or a combination thereof, or other biologically acceptable pH buffered solutions. In some embodiments, an extracellular matrix gel comprises a tissue-derived stimuli-responsive gel. In some embodiments, the multi-component fluid comprises a smart material which may exhibit responsiveness to external stimuli including temperature, pH, ionic concentration, light, magnetic fields, electrical fields, chemicals, or enzymes.

In some embodiments, the multi-component fluid may be delivered to prevent post-operative adhesions. In some embodiments, the systems and method comprise mixing two fluids together within a spray device to form the multi-component fluid. In some embodiments, the multi-component fluid may be delivered through a nozzle as particles or droplets having a maximum dimension of 500 μm. In some cases, the particles have a dimension of about 10 μm to about 500 μm. In some embodiments, the particles have a maximum dimension of 300 μm. In some cases, the particles have a dimension of about 10 μm to about 20 μm, about 10 μm to about 30 μm, about 10 μm to about 50 μm, about 10 μm to about 100 μm, about 10 μm to about 150 μm, about 10 μm to about 200 μm, about 10 μm to about 300 μm, about 10 μm to about 400 μm, about 10 μm to about 500 μm, about 20 μm to about 30 μm, about 20 μm to about 50 μm, about 20 μm to about 100 μm, about 20 μm to about 150 μm, about 20 μm to about 200 μm, about 20 μm to about 300 μm, about 20 μm to about 400 μm, about 20 μm to about 500 μm, about 30 μm to about 50 μm, about 30 μm to about 100 μm, about 30 μm to about 150 μm, about 30 μm to about 200 μm, about 30 μm to about 300 μm, about 30 μm to about 400 μm, about 30 μm to about 500 μm, about 50 μm to about 100 μm, about 50 μm to about 150 μm, about 50 μm to about 200 μm, about 50 μm to about 300 μm, about 50 μm to about 400 μm, about 50 μm to about 500 μm, about 100 μm to about 150 μm, about 100 μm to about 200 μm, about 100 μm to about 300 μm, about 100 μm to about 400 μm, about 100 μm to about 500 μm, about 150 μm to about 200 μm, about 150 μm to about 300 μm, about 150 μm to about 400 μm, about 150 μm to about 500 μm, about 200 μm to about 300 μm, about 200 μm to about 400 μm, about 200 μm to about 500 μm, about 300 μm to about 400 μm, about 300 μm to about 500 μm, or about 400 μm to about 500 μm. In some cases, the particles have a dimension of about 10 μm, about 20 μm, about 30 μm, about 50 μm, about 100 μm, about 150 μm, about 200 μm, about 300 μm, about 400 μm, or about 500 μm. In some cases, the particles have a dimension of at least about 10 μm, about 20 μm, about 30 μm, about 50 μm, about 100 μm, about 150 μm, about 200 μm, about 300 μm, or about 400 μm. In some cases, the particles have a dimension of at most about 20 μm, about 30 μm, about 50 μm, about 100 μm, about 150 μm, about 200 μm, about 300 μm, about 400 μm, or about 500 μm. In some embodiments, the particles have a dimension of more than 500 μm or less than 10 μm.

The ability of the multi-component fluid or gel to adhere to the application site (e.g. the yield stress) can be an important factor in its ability to prevent post-surgical adhesions. Yield stress can be an indicator of the internal strength of the gel. As load is applied to the gel, stress can increase, and if the stress exceeds the strength of the gel, the gel may yield or fracture. This can happen at either of 2 places: (a) the interface between the gel and the tissue where it is applied, or (b) within the gel itself. If the gel fractures within the gel, that can leave a coating on the tissue surface. In some cases, the adhesive strength of the gel to the tissue is greater than the internal cohesiveness of the gel.

In some cases, the yield stress of the multi-component fluid or gel described herein is between about 100 Pa and 1000 Pa. The yield stress can be between about 100 Pa to about 200 Pa, about 100 Pa to about 300 Pa, about 100 Pa to about 400 Pa, about 100 Pa to about 500 Pa, about 100 Pa to about 600 Pa, about 100 Pa to about 700 Pa, about 100 Pa to about 800 Pa, about 100 Pa to about 900 Pa, about 100 Pa to about 1000 Pa, about 200 Pa to about 300 Pa, about 200 Pa to about 400 Pa, about 200 Pa to about 500 Pa, about 200 Pa to about 600 Pa, about 200 Pa to about 700 Pa, about 200 Pa to about 800 Pa, about 200 Pa to about 900 Pa, about 200 Pa to about 1000 Pa, about 300 Pa to about 400 Pa, about 300 Pa to about 500 Pa, about 300 Pa to about 600 Pa, about 300 Pa to about 700 Pa, about 300 Pa to about 800 Pa, about 300 Pa to about 900 Pa, about 300 Pa to about 1000 Pa, about 400 Pa to about 500 Pa, about 400 Pa to about 600 Pa, about 400 Pa to about 700 Pa, about 400 Pa to about 800 Pa, about 400 Pa to about 900 Pa, about 00 Pa to about 1000 Pa, about 500 Pa to about 600 Pa, about 500 Pa to about 700 Pa, about 500 Pa to about 800 Pa, about 500 Pa to about 900 Pa, about 500 Pa to about 1000 Pa, about 600 Pa to about 700 Pa, about 600 Pa to about 800 Pa, about 600 Pa to about 900 Pa, about 600 Pa to about 1000 Pa, about 700 Pa to about 800 Pa, about 700 Pa to about 900 Pa, about 700 Pa to about 1000 Pa, 800 Pa to about 900 Pa, about 800 Pa to about 1000 Pa, and between about 900 Pa to about 1000 Pa. In some cases, the yield stress is between 200 Pa and 700 Pa. The yield stress can be between about 200 Pa to about 300 Pa, about 200 Pa to about 400 Pa, about 200 Pa to about 500 Pa, about 200 Pa to about 600 Pa, about 200 Pa to about 700 Pa, about 300 Pa to about 400 Pa, about 300 Pa to about 500 Pa, about 300 Pa to about 600 Pa, about 300 Pa to about 700 Pa, about 400 Pa to about 500 Pa, about 400 Pa to about 600 Pa, about 400 Pa to about 700 Pa, about 500 Pa to about 600 Pa, about 500 Pa to about 700 Pa, and between about 600 Pa to about 700 Pa. The yield stress can be between about 200 Pa to about 400 Pa.

In some cases, the yield stress can be less than about 100 Pa, less than about 200 Pa, less than about 300 Pa, less than about 400 Pa, less than about 500 Pa, less than about 600 Pa, less than about 700 Pa, less than about 800 Pa, less than about 900 Pa, or less than about 1000 Pa, In some cases, the yield stress can be greater than about 100 Pa, greater than about 200 Pa, greater than about 300 Pa, greater than about 400 Pa, greater than about 500 Pa, greater than about 600 Pa, greater than about 700 Pa, greater than about 800 Pa, greater than about 900 Pa, or greater than about 1000 Pa,

The yield stress can vary depending on the presence or absence of tissue on, or to, which the multi-component fluid or a gel adheres. In some cases, the yield stress is greater when there is no tissue to adhere to. In some cases, the yield stress is between about 150 Pa and 250 Pa in the presence of tissue. In some cases, the yield stress is between about 100 Pa and 700 Pa in the absence of tissue.

### Housing Tube

As described herein, in some embodiments, an application device comprises a housing tube. In some embodiments, the tube may be in fluidic communication with one or more containers, as described herein. In some embodiments, the tube may be configured to deliver one or more fluid components from the one or more containers to an outlet of the spray device (e.g., a tube opening, mixer, a nozzle). In some embodiments, the housing has multiple lumens. A multi-lumen housing may have at least 2, 3, 4, 5, 6, 7, 8, 9, 10, or more lumens. In some embodiments, the multi-lumen housing may have at most about 10, 9, 8, 7, 6, 5, 4, 3, or 2 lumens. The housing tube can comprise a dual barrel cartridge holder. In some embodiments, the one or more lumens of the multi-lumen housing may be formed through extrusion. In some embodiments, the extruded lumens have increased elastic moduli, a highly uniform cross-sectional area, increased flexibility, increased mechanical properties, are smooth along the lateral surface of the lumen, and have a low coefficient of friction. One or more lumens of a multi-lumen housing may carry a different fluid component of the multi-component fluid. In some embodiments, two or more of the lumens of a multi-lumen housing may have a similar cross-section or they may have a different cross-section. The cross-sectional area of two or more lumens may be a ratio of approximately 1:1, 2:1, 3:1, 4:1, 5:1, 6:1, 7:1, 8:1, 9:1, 10:1, or greater than 10:1. In some embodiments, each lumen of the multi-lumen may be configured to span a portion of a housing length. In some embodiments, each lumen spans at least about 60% to about 100% of the housing length. In some embodiments, each lumen spans from a proximal end of the housing to a location distal to the distal end of the housing. In some embodiments, one or more lumens of the multi-lumen housing spans a different length from the other lumens.

In some embodiments, the multi-lumen housing has a length of between about 1 centimeter (cm) to about 40 cm. In some embodiments, a multi-lumen housing may have a length of about 1 cm to about 5 cm, about 1 cm to about 10 cm, about 1 cm to about 15 cm, about 1 cm to about 20 cm, about 1 cm to about 30 cm, about 1 cm to about 40 cm, about 5 cm to about 10 cm, about 5 cm to about 15 cm, about 5 cm to about 20 cm, about 5 cm to about 30 cm, about 5 cm to about 40 cm, about 10 cm to about 15 cm, about 10 cm to about 20 cm, about 10 cm to about 30 cm, about 10 cm to about 40 cm, about 15 cm to about 20 cm, about 15 cm to about 30 cm, about 15 cm to about 40 cm, about 20 cm to about 30 cm, about 20 cm to about 40 cm, or between about 30 cm to about 40 cm. In some embodiments, a multi-lumen housing may have a length of about 1 cm, about 5 cm, about 10 cm, about 15 cm, about 20 cm, about 25 cm, about 30 cm, about 35 cm, or about 40 cm. In some embodiments, the multi-lumen housing has a length of about 30 cm to about 40 cm. In some embodiments, a multi-lumen housing may have a length of at least about 1 cm, about 5 cm, about 10 cm, about 20 cm, about 30 cm, or about 40 cm. In some embodiments, a multi-lumen housing may have a length of at most about 40 cm, about 30 cm, about 20 cm, about 10 cm, about 5 cm, about 1 cm, or less.

In some embodiments, the outer diameter of the multi-lumen housing may be varied to accommodate various surgical port sizes. The outer diameter of the multi-lumen housing may be about 1 millimeter (mm) to about 15 mm. The outer diameter of the housing may be about: 1 mm, 2 mm, 3 mm, 4 mm, 5 mm, 6 mm, 7 mm, 8 mm, 9 mm, 10 mm, or more than 15 mm. In some embodiments, the outer diameter of the housing may be a diameter between any of the two diameters mentioned herein or a diameter less than 1 mm. In some embodiments, the outer diameter of the multi-lumen housing may be between 5 mm to about 12 mm, about 5 mm to about 8 mm, about 8 mm to about 12 mm. In some embodiments, the outer diameter of the multi-lumen housing may be 5 mm, 8 mm, or 12 mm. In some cases, the outer diameter can be between about 5 mm and about 6 mm. In some cases, the outer diameter can be about 5 mm, about 5.1 mm, about 5.2 mm, about 5.3 mm, about 5.4 mm, about 5.5 mm, about 5.6 mm, about 5.7 mm, about 5.8 mm, about 5.9 mm, or about 6 mm. In some cases, the outer diameter can be about 5.5 mm.

In some embodiments, the housing comprises a housing opening located at a distal end. In some embodiments, the housing opening may be configured as a nozzle as described herein. In some embodiments, the housing opening may be coupled to a nozzle as described herein. In some embodiments, the housing opening may be coupled to a mixer. In some embodiments, as described herein, a mixer may be disposed within the housing and located distal to the housing opening. In some embodiments, the mixer may be disposed within a distal end of the housing and located proximal to the housing opening or a nozzle.

In some embodiments, the housing may be coupled to a nozzle, mixer, or other components using an adhesive. An adhesive may comprise a structural adhesive, pressure sensitive adhesive, thermosetting adhesive, epoxy, polyurethane, polyimides, paste, liquid, film, pellet, tape, hot melt adhesive, contact adhesive, reactive hot melt adhesive, cyanoacrylate, urethanes, acrylics, glue, resin, anaerobic, cyanoacrylate glue, hot glue, polyvinyl acetate, silicones, phenolics, instant glues, plastisols or another chemical joint. In some embodiments, the multi-lumen housing may be coupled to a mixer, nozzle, other components using barbed tubing fittings or other mechanical joint. In some embodiments, the multi-lumen housing may be coupled to a mixer, nozzle, or other components using a weld. In some embodiments, the weld may be performed using ultrasonic welding, thermal welding, or other method of fusing. In some embodiments, the multi-lumen housing may be coupled to a mixer, nozzle, or other components using threads. In some embodiments, the threads can comprise polymeric or metallic threads. In some cases, the threads can comprise polycarbonate, stainless steel, nylon, polyester, polytetrafluoroethylene threads, or any combination thereof. In some embodiments, the multi-lumen housing may be affixed to adjacent components with adhesive, threads, barbed tubing fittings, a weld, or any combination thereof.

### Mixer

In some embodiments, the application device comprises a mixer. The mixer may be configured to mix two or more fluid components of the multi-component fluid. In some embodiments, the mixer may be configured to mix an extracellular matrix pre-gel material (ECM) and pH buffer. The mixed fluid may form a homogeneous fluid after being mixed by the mixer. In some embodiments, the mixer may be a static mixer. In some embodiments, the mixer may be within the tube. In some embodiments, the mixer may be distal to the tube end or a distal portion of the tube thereof. In some embodiments, a nozzle may be located within the mixer and distal to the mixer body. In some embodiments, the mixer may be configured to receive two or more fluid components from the multi-lumen tube and mix the two or more fluid components. In some embodiments, one or more lumens terminate at or proximal to the mixer. In some embodiments, the fluid components are driven towards the nozzle by the pressure provided from a driving force attached to the fluid containers (e.g., the plunger in the plunging system), a force generated by the movement of the mixer, or a combination of both. The mixer may substantially completely mix the two or more fluid components (e.g., a substantially homogenized mix) prior to the multi-component fluid being delivered from the spray device.

In some embodiments, a mixer comprises a mixer body. In some embodiments, the mixer body comprises a central shaft. The mixer body may comprise one or more mixing elements or stages. In some embodiments, the one or more mixing elements may be attached to the central shaft. The one or more mixing elements may comprise a spacer, a blade, a fin, a semi-circular cylinder, or a channel. In some embodiments, the one or more mixing elements may comprise the central shaft. In some embodiments, the mixer receives one or more fluids from the multi-lumen tube. In some embodiments, the mixer comprises an annular cavity.

### Aerosolization

In some embodiments, the application device aerosolizes the mixture (e.g., multi-component fluid) prior to or during delivery to apply it as a spray. Aerosolization may improve uniformity of the application of the multi-component fluid to an area of interest. In some embodiments, the dispersant (e.g., dispersant gas, as described herein) may facilitate aerosolization of the multi-component fluid. In some embodiments, the dispersant facilitates aerosolization by generating a pressure difference between the nozzle inlet and the nozzle outlet. In some embodiments, the dispersant may be provided to the nozzle via the dispersant passageway within the central shaft of the mixer body (as described herein). In some embodiment, the dispersant from the dispersant passageway contacts the multi-component fluid (e.g., formed via the mixer as described herein) prior to, within, or downstream the nozzle. In some embodiments, the dispersant from the dispersant passageway contacts the multi-component fluid (e.g., formed via the mixer as described herein) prior to, within, or downstream the nozzle outlet. The dispersant may be configured to carry particles (e.g., droplets) of the multi-component fluid after delivery from the nozzle outlet. In some embodiments, nozzle shape or a geometrical feature which may protrude into or be cut out of the nozzle orifice may perturb the flow of the multicomponent fluid as it exits the nozzle orifice to facilitate aerosolization. The material to be sprayed may be sensitive to high shear stress. A mixer or a nozzle may be configured to mitigate, obviate, or induce shear stress by geometric features. In some embodiments, aerosolization may be facilitated by injection of a dispersant (e.g., a compressed gas). The multi-component fluid may be delivered through the nozzle outlet in form of a jet of fluid. In some embodiments, a dispersant can be delivered through the nozzle simultaneously with the multifluid delivery out of the nozzle outlet to form aerosols.

### Devices

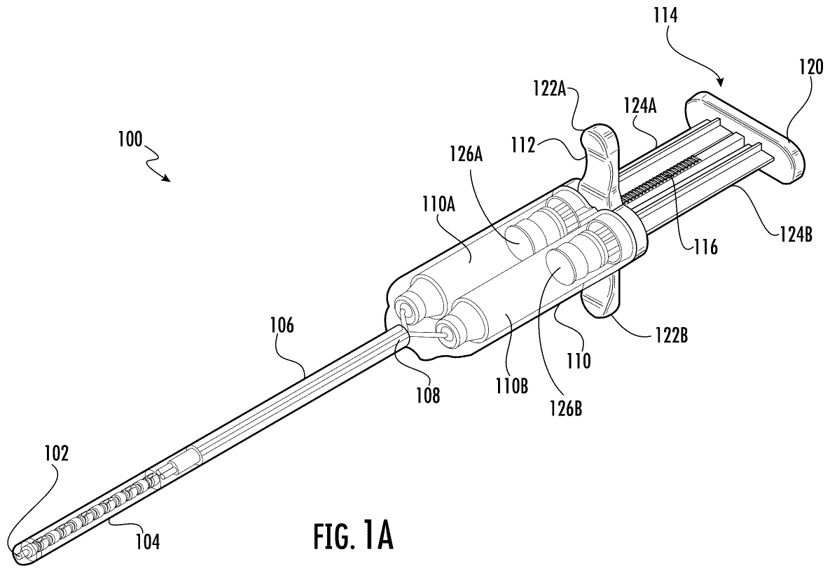

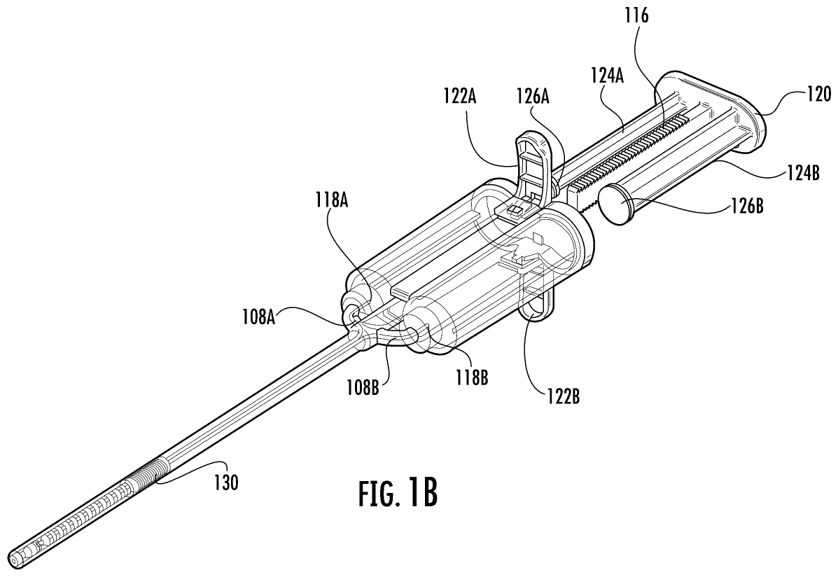

FIG. 1A shows a perspective view of an exemplary embodiment of a delivery device as described herein. Device 100 for delivery of a multi-component fluid can comprise precision nozzle 102, mixing attachment 104, arthroscopic extension 106, piercing hypotubes 108, dual barrel cartridge holder 110, finger grip 112, ratcheted dispenser 114, and thumb grip 120, or any combination thereof. The nozzle 102 can be the most distal part of device 100. In some cases, the mixer attachment and/or mixer 104 is proximal to the nozzle 102. The arthroscopic extension 106 can be proximal to the mixer 104. The dual barrel cartridge holder 110 can be proximal to the arthroscopic extension 106. The dual barrel cartridge holder 110 can include a first cartridge or barrel 110A and a second cartridge or barrel 110B, each of which has a lumen. The dual barrel cartridge holder 110 can meet the arthroscopic extension 106 at the piercing hypotubes 108. The finger grip 112 can be proximal to the dual barrel cartridge holder 110 and comprise first extension 122A

and second extension 122B. The first and second extensions 122A, 122B can extend in opposite directions to one another, as shown in FIG. 1. Each of the first and second extensions 122A, 122B can include a grooved, curved or contoured surface for ease of finger placement. For example, as shown in FIG. 1B, the surface can include ribs or ridges to improve handling. The ratcheted dispenser 114 can be proximal to the finger grip 112 and include a series of teeth 116 for engagement with corresponding teeth of the finger grip 112 to allow controlled, step-wise ratcheting when depressed. This ratcheted dispenser 114 serves as a plunger into the double barrel cartridge holder 110, and include first and second legs 124A, 124B.

As shown in greater detail in FIG. 1B, the first leg 124A extends into a first push pad 126A, while second leg 124B extends into a second push pad 126B. The push pads 126A, 126B are configured to slide inside the first barrel 110A and second barrel 110B when the ratcheting dispenser 114 is depressed against the double barrel cartridge holder 110. The ratcheting dispenser 114 can include a plunger top 120, or thumb grip, against which the user can apply pressure to effect the plunging motion. The thumb grip 120 can be proximal and coupled to the ratcheted dispenser 114. The piercing hypotubes 108 can include a first piercing end 118A that is in communication and/or extends into the first barrel or cartridge 110A, and a second piercing end 118B that is in communication and/or extends into the second barrel or cartridge 110B. The nozzle 102, mixer 104, an optional additional fluid mixing component 130, arthroscopic extension 106, piercing hypotubes 108, and dual barrel cartridge holder 110 can be fluidly coupled to the proximal and distal element.

The device 100 can be sized to be held in one hand. The device 100 can be sized to be held in two hands. The device 100 can be sized to be held with three fingers. In some cases, the device 100 can be used with two fingers (e.g. one finger and a thumb). In some cases, the length of device 100 can be between about 10 mm and about 60 mm. In some cases, the length of device 100 can be between about 10 mm to about 15 mm, about 10 mm to about 20 mm, about 10 mm to about 25 mm, about 10 mm to about 30 mm, about 10 mm to about 35 mm, about 10 mm to about 40 mm, about 10 mm to about 45 mm, about 10 mm to about 50 mm, about 10 mm to about 55 mm, about 10 mm to about 60 mm, about 15 mm to about 20 mm, about 15 mm to about 25 mm, about 15 mm to about 30 mm, about 15 mm to about 35 mm, about 15 mm to about 40 mm, about 15 mm to about 45 mm, about 15 mm to about 50 mm, about 15 mm to about 55 mm, about 15 mm to about 60 mm, about 20 mm to about 25 mm, about 20 mm to about 30 mm, about 20 mm to about 35 mm, about 20 mm to about 40 mm, about 20 mm to about 45 mm, about 20 mm to about 50 mm, about 20 mm to about 55 mm, about 20 mm to about 60 mm, about 25 mm to about 30 mm, about 25 mm to about 35 mm, about 25 mm to about 40 mm, about 25 mm to about 45 mm, about 25 mm to about 50 mm, about 25 mm to about 55 mm, about 25 mm to about 60 mm, about 30 mm to about 35 mm, about 30 mm to about 40 mm, about 30 mm to about 45 mm, about 30 mm to about 50 mm, about 30 mm to about 55 mm, about 30 mm to about 60 mm, about 35 mm to about 40 mm, about 35 mm to about 45 mm, about 35 mm to about 50 mm, about 35 mm to about 55 mm, about 35 mm to about 60 mm, about 40 mm to about 45 mm, about 40 mm to about 50 mm, about 40 mm to about 55 mm, about 40 mm to about 60 mm, about 45 mm to about 50 mm, about 45 mm to about 55 mm, about 45 mm to about 60 mm, about 50 mm to about 55 mm, about 50 mm to about 60 mm, about 55 mm to about 60 mm. In some cases, the length of the device 100 is at least about 10 mm, at least about 15 mm, at least about 20 mm, at least about 25 mm, at least about 30 mm, at least about 35 mm, at least about 40 mm, at least about 45 mm, at least about 50 mm, at least about 55 mm, or at least about 60 mm.

In some embodiments, the length of device 100 can be between about 1 centimeter (cm) to about 40 cm. In some embodiments, device 100 may have a length of about 1 cm to about 5 cm, about 1 cm to about 10 cm, about 1 cm to about 15 cm, about 1 cm to about 20 cm, about 1 cm to about 30 cm, about 1 cm to about 40 cm, about 5 cm to about 10 cm, about 5 cm to about 15 cm, about 5 cm to about 20 cm, about 5 cm to about 30 cm, about 5 cm to about 40 cm, about 10 cm to about 15 cm, about 10 cm to about 20 cm, about 10 cm to about 30 cm, about 10 cm to about 40 cm, about 15 cm to about 20 cm, about 15 cm to about 30 cm, about 15 cm to about 40 cm, about 20 cm to about 30 cm, about 20 cm to about 40 cm, or between about 30 cm to about 40 cm. In some embodiments, device 100 may have a length of about 1 cm, about 5 cm, about 10 cm, about 15 cm, about 20 cm, about 25 cm, about 30 cm, about 35 cm, or about 40 cm. In some embodiments, device 100 has a length of about 30 cm to about 40 cm. In some embodiments, device 100 may have a length of at least about 1 cm, about 5 cm, about 10 cm, about 15 cm, about 20 cm, about 30 cm, or about 40 cm. In some embodiments, device 100 may have a length of at most about 40 cm, about 30 cm, about 20 cm, about 15 cm, about 10 cm, about 5 cm, about 1 cm, or less.

The widest part of the thumb grip 120 can be parallel to the widest part of the finger grip 112. The widest part of the thumb grip 120 can be perpendicular to the widest part of the finger grip 112. The thumb grip 120 can be wider than the finger grip 112. The thumb grip 120 can be narrower than the finger grip 112. The thumb grip and finger grip can have a width between about 5 mm to about 20 mm. The width can be less than about 5 mm, less than about 10 mm, less than about 15 mm, or less than about 20 mm. The width can be greater than about 5 mm, greater than about 10 mm, greater than about 15 mm, or greater than about 20 mm.

The ratcheted dispenser 114 can comprise a piston, plunger, pump, multi-step button, or any other type of actuator. With respect to FIG. 1, the ratcheted dispenser can comprise a piston where the actuated part is between the finger grip 112 and the thumb grip 120 while the piston seal is inside the dual barrel cartridge holder 110.

The dual barrel cartridge holder 110 comprises two cartridges. In some cases, a cartridge holder can comprise 1, 2, 3, 4, or more cartridges depending on the number of fluids to be mixed to create the multi-component fluid. The cartridges can be loaded with one or more fluids. In some cases, the cartridges are loaded with the same fluid. In some cases, they are loaded with different fluid. In some cases, one or more cartridges can be loaded with an extracellular matrix (ECM). In some embodiments, one or more cartridges can be loaded with a buffer. The buffer can be a phosphate buffered saline. The ECM can comprise a mildly acidic pH. The pH can be from about 6.5 to about 7.0. The pH can be less than 6.5, less than 6.6, less than 6.7, less than 6.8, less than 6.9, or less than 7.0. The pH can be greater than 6.5, greater than 6.6, greater than 6.7, greater than 6.8, greater than 6.9, or greater than 7.0.

The buffer can be mildly basic. The pH can be from about 7.5 to about 8.0. The pH can be less than 7.5, less than 7.6, less than 7.7, less than 7.8, less than 7.9, or less than 8.0. The pH can be greater than 7.5, greater than 7.6, greater than 7.7, greater than 7.8, greater than 7.9, or greater than 8.0.

The multi-component fluid can be mildly acidic. The pH can be from about 6.5 to about 7.0. The pH can be less than 6.5, less than 6.6, less than 6.7, less than 6.8, less than 6.9, or less than 7.0. The pH can be greater than 6.5, greater than 6.6, greater than 6.7, greater than 6.8, greater than 6.9, or greater than 7.0.

The diameter of these cartridges can be the same. The diameter can be unequal. The diameter of these cartridges can be between about 5 mm to about 20 mm. The diameter of these cartridges can be between about 5 mm to about 10 mm, about 5 mm to about 15 mm, about 5 mm to about 20 mm, about 10 mm to about 15 mm, about 10 mm to about 20 mm, or between about 15 mm to about 20 mm. The diameter can be less than about 5 mm, less than about 10 mm, less than about 15 mm, or less than about 20 mm. The diameter can be greater than about 5 mm, greater than about 10 mm, greater than about 15 mm, or greater than about 20 mm. In some cases, the diameter can be about 6.85 mm, about 8.85 mm, about 12 mm, or about 14 mm.

In some cases, the inner diameter of the cartridges can be between about 5 mm and about 13 mm. In some cases, the inner diameter of the cartridges can be between about 5 mm to about 7 mm, about 5 mm to about 9 mm, about 5 mm to about 11 mm, about 5 mm to about 13 mm, about 7 mm to about 9 mm, about 7 mm to about 11 mm, about 7 mm to about 13 mm, about 9 mm to about 11 mm, about 9 mm to about 13 mm, or between about 11 mm to about 13 mm. The inner diameter can be at least about 5 mm, at least about 7 mm, at least about 9 mm, at least about 11 mm, or at least about 13 mm. The inner diameter can be at most about 5 mm, at most about 7 mm, at most about 9 mm, at most about 11 mm, or at most about 13 mm. In some cases, the inner diameter of the cartridges can be between about 6.85 mm and about 12 mm. In some cases, the inner diameter of the cartridges can be about 6.85 mm or about 12 mm.

In some cases, the outer diameter of the cartridges can be between about 7 mm and about 15 mm. In some cases, the outer diameter of the cartridges can be between about 7 mm to about 9 mm, about 7 mm to about 11 mm, about 7 mm to about 13 mm, about 7 mm to about 15 mm, about 9 mm to about 11 mm, about 9 mm to about 13 mm, about 9 mm to about 15 mm, about 11 mm to about 13 mm, about 11 mm to about 15 mm, or between 13 mm to about 15 mm. The outer diameter can be at least about 7 mm, at least about 9 mm, at least about 11 mm, at least about 13 mm, or at least about 15 mm. The outer diameter can be at most about 7 mm, at most about 9 mm, at most about 11 mm, at most about 13 mm, or at most about 15 mm. In some cases, the outer diameter of the cartridges can be between about 8.85 mm and about 14 mm. In some cases, the outer diameter of the cartridges can be about 8.85 mm or about 14 mm.

In some cases, one or more fluids is inserted into one or more of the cartridges and the cartridge is inserted into the cartridge holder. In some cases, the cartridge remains in fluid communication with a separate fluid container. In some cases, the cartridge acts as a fluid container for downstream processes (e.g., mixing and/or dispensing).

The piercing hypotubes 108 fluidly couple the cartridges in the dual barrel cartridge holder 110 to the arthroscopic extension 106. One hypotube can pierce one cartridge so that the fluids do not mix at this stage. In some cases, the fluids can begin mixing. The arthroscopic extension 106 and piercing hypotubes 108 impart shear stress on the fluids moving through them by narrowing the fluid path. The arthroscopic extension 106 is physically and fluidly coupled to the mixer.

The delivery device may comprise a mixer 104 disposed distal to arthroscopic extension 106 and proximal to the nozzle 102. In some cases, the fluids from the separate hypotubes combine when they reach mixer 104, thereby automatically creating some amount of mixing. In some cases, the mixer design, as discussed below, encourages mixing by limiting the fluid domain (e.g. where the fluid can reside). By narrowing the fluid domain, the mixer imparts additional shear stress on the fluid to increase the yield stress of the resulting gel. The application of shear stress through mixing can alter the viscosity. Delivery can comprise applying the multi-component fluid to a location in need (e.g., a wound or open incision site) with device 100. Applying the multi-component fluid can be beneficial when a slow and careful application is desirable. In some cases, the multi-component fluid is viscous. The viscosity of the multi-component fluid can range from about 5 centipoise (cP) to about 1,000,000 cP (1 cP=1 mPa*s) as discussed above.

The mixer can comprise multiple stages. The mixer can comprise an array of stages. In some cases, the distal end of the mixer can comprise one or more vortex inducers. The one or more vortex inducers can impose shear stress on the multi-component fluid to generate a high yield stress (e.g. between about 200 Pa and about 700 Pa) of the resulting gel as discussed above. Shear stress can be derived using a computational model. The computational model can use artificial intelligence or machine learning computing. In some cases, the vortex inducers can also impose rotation or swirl (e.g. vorticity) on the multi-component liquid.

In some cases, the mixer 104 can comprise one or more spacers. The spacer can be located between vortex inducers. In some cases, the distal end of a mixer 104 comprises a vortex inducer, a spacer, and a second vortex inducer. There may be multiple iterations of vortex inducer, spacer, vortex inducer to impart further shear stress on the multi-component fluid. In some cases, there may be multiple vortex inducers in a row without a spacer in between.

In some cases, the mixer 104 does not comprise one or more vortex inducers. In some cases, the mixer 104 does not comprise one or more spacers. The mixer 104 can be fluidly coupled to the precision nozzle outlet and/or the outlet of device 100.

The delivery device may comprise a nozzle 102 disposed distal to the mixer 104. The nozzle may comprise a nozzle inlet, a nozzle body, and a nozzle outlet. The nozzle can receive the multi-component fluid from the mixer and deliver the multi-component fluid through the nozzle outlet to a location external to the device 100. In some cases, delivery can comprise dispersing the multi-component fluid (e.g. when the multi-component fluid is more liquid, such that it can be applied or dispensed). The application of shear stress through mixing can increase the viscosity. In some cases, the multi-component fluids delivered from the delivery device may be non-Newtonian fluids. The types of non-Newtonian fluids can be shear-thinning or shear-thickening fluids that decrease or increase in viscosity, respectively, based on the application of shear stress through mixing. Delivery can comprise applying the multi-component fluid to a location in need (e.g., a wound or open incision site) with device 100. Applying the multi-component fluid can be beneficial when a slow and careful application is desirable. In some cases, the multi-component fluid is viscous. The viscosity of the multi-component fluid can range from about 5 centipoise (cP) to about 1,000,000 cP (1 cP=1 mPa*s) as discussed above.

In some cases, one or more of the nozzle inlet, nozzle body, or nozzle outlet can comprise one or more vortex inducers. The one or more vortex inducers can impose shear stress on the multi-component fluid to generate a high yield stress (e.g. between about 200 Pa and about 700 Pa) of the resulting gel as discussed above. Shear stress can be derived using a computational model. The computational model can use artificial intelligence or machine learning computing. In some cases, the vortex inducers can also impose rotation or swirl (e.g. vorticity) on the multi-component liquid.

In some cases, one or more of the nozzle inlet, nozzle body, or nozzle outlet can comprise one or more spacers. The spacer can be located between vortex inducers. In some cases, the nozzle comprises a nozzle inlet comprising a vortex inducer, a nozzle body comprising a spacer, and a nozzle outlet comprising a second vortex inducer. There may be multiple iterations of vortex inducer, spacer, vortex inducer to impart further shear stress on the multi-component fluid. In some cases, there may be multiple vortex inducers in a row without a spacer in between. In some cases, the entirety of the nozzle comprises a vortex inducer without a spacer.

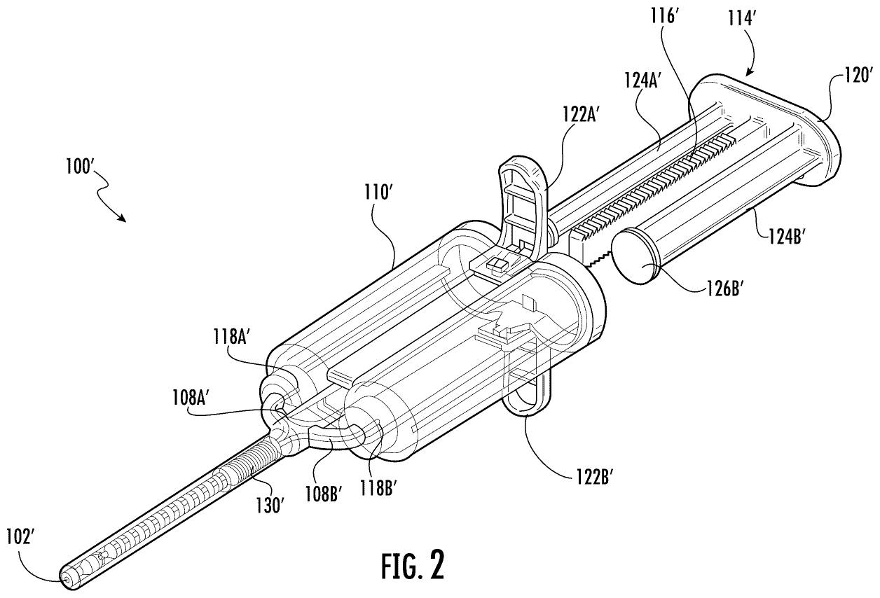

FIG. 2 illustrates another exemplary embodiment of a delivery device 100′ as described herein. Device 100′ is similar to device 100 of FIGS. 1A and 1B, wherein similar elements have the same reference number followed by the symbol “′”, except that the arthroscopic extension 106′ and hypotubes 108′ of device 100′ are shorter than arthroscopic extension 106 and hypotube 108 of device 100.

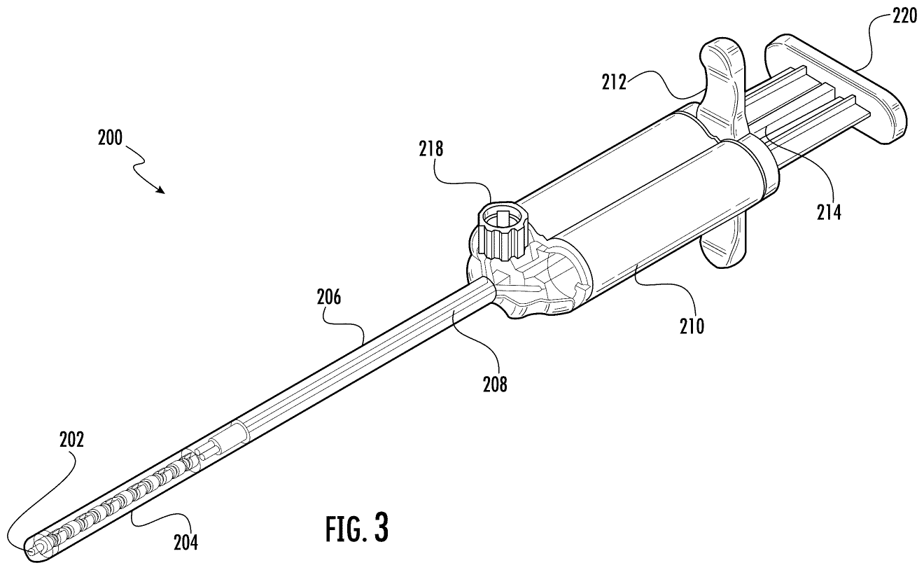

FIG. 3 depicts a perspective view of another exemplary embodiment of a delivery device as described herein. Device 200 comprises a precision nozzle 202, a mixing attachment 204, an arthroscopic extension 206, piercing hypotubes 208, a dual barrel cartridge holder 210, finger grip 212, ratcheting dispenser 214, thumb grip 220, and gas-assist luer port 218. Device 200 is similar to device 100 with the exception of the addition of gas-assist luer port 218.

The gas-assist luer port 218 can act as an entryway for gas into an otherwise closed system device 200. Gas can enter by being directly attached to a gas source or fluidly connected through a gas tube. The gas can be compressed gas. In some embodiments, the compressed gas may comprise oxygen, carbon dioxide, Nitrogen, helium, atmospheric air, argon, neon, xenon, krypton, radon, acetylene, butane, ethylene, hydrogen, methylamine, vinyl chloride, nitrogen oxides, halogen gases (e.g., chlorine, fluorine), acetylene,1,3-butadiene, methyl acetylene, tetrafluoroethylene, vinyl fluoride, or combinations thereof. The gas can enter at the distal tip of the mixer to disperse or aerosolize the gel or liquids. In some cases, a gas source can be a gas supplying machine, a container that requires mechanical compression, etc. In some cases, a steady flow of gas can be supplied through the gas-assist luer port. In some cases, the amount of compressed gas can be regulated by the device that supplies the gas.

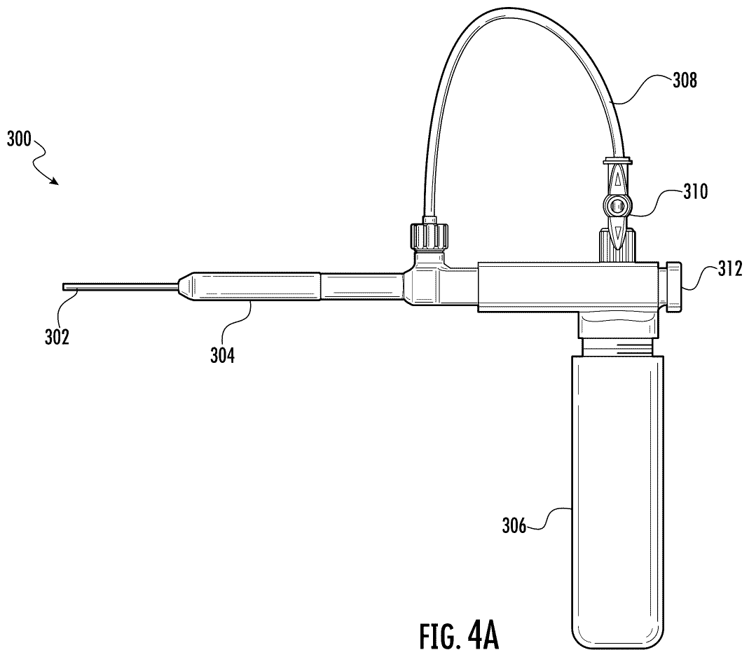

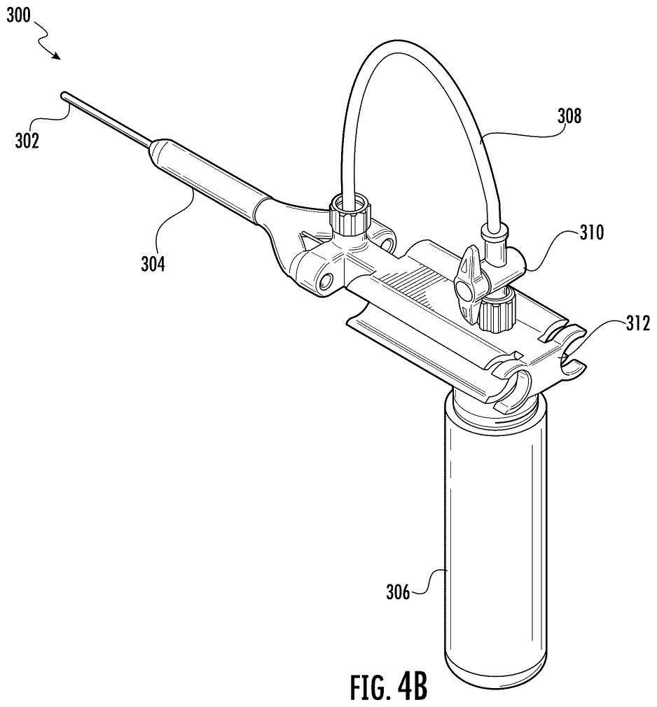

FIGS. 4A and 4B depict side and perspective top views, respectively, of another exemplary embodiment of a delivery device as described herein. Device 300 comprises a nozzle tip 302, a mixer 304, a gas cylinder 306, a gas tube 308, a gas valve 310, and a syringe holder 312. Unlike in devices 100 and 200, a user can hold the device at the gas cylinder 306 instead of the syringe part of the device. Nozzle tip 302 and mixer 304 can be similar to devices 100 and 200. Nozzle tip 302 and mixer 304 can have one or more vortex inducers and one or more spacers. Nozzle tip 302 and mixer 304 can only have mixing components. The mixing components can be similar to the mixing components described below. The mixing components can comprise a spacer, a blade, a fin, or a channel.

As in device 200, device 300 of FIG. 4B uses gas-assisted pressure to disperse or aerosolize the gel or liquids. The gas can be compressed. The gas can comprise carbon dioxide. The gas can comprise oxygen, carbon dioxide, Nitrogen, helium, atmospheric air, argon, neon, xenon, krypton, radon, acetylene, butane, ethylene, hydrogen, methylamine, vinyl chloride, nitrogen oxides, halogen gases (e.g., chlorine, fluorine), acetylene,1,3-butadiene, methyl acetylene, tetrafluoroethylene, vinyl fluoride, or combinations thereof.

The amount and flow of gas can be regulated through gas valve 310. The valve can be rotated to allow more or less gas through. The valve can allow gas through faster or slower. The gas can come from gas cylinder 306. Gas valve 310 can connect gas tube 308 with gas cylinder 306. Gas cylinder 306 can comprise rigid metal. Gas cylinder 306 can be held by a user. The user can regulate the amount, flow, and speed of gas through adjusting the valve.

Syringe holder 312 can act as a physical and fluid intermediary between the gas valve 310, gas tube 308, gas cylinder 306, and the device cartridges. The semi-circular grasping empty areas in syringe holder 312 as shown in FIG. 4B can be used to insert device cartridges comprising fluids.

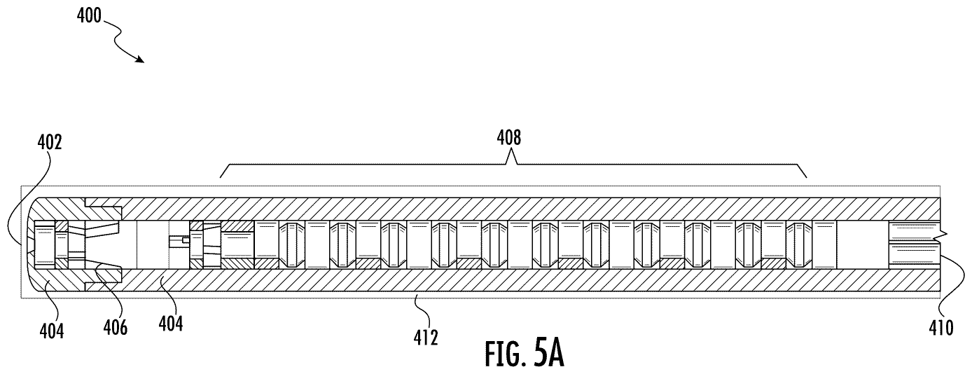

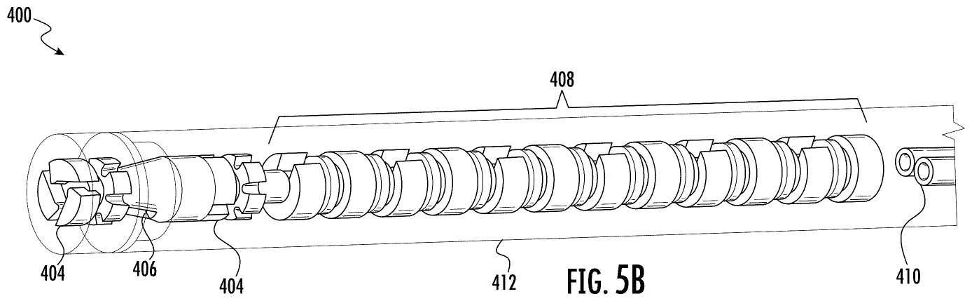

FIG. 5A depicts an exemplary embodiment of an expanded side view of the mixing element shown in an exemplary embodiment of a delivery device as depicted herein. FIG. 5B depicts an example embodiment of an expanded perspective view of the mixing element shown in an exemplary embodiment of a delivery device as depicted herein. Mixer 400 is an example of a mixer as described herein. Mixer 400 comprises nozzle tip 402, vortex inducers 404, spacer 406, and mixer array 408. Piercing hypotubes 410 from an arthroscopic extension are shown proximal to the mixer array 408. A series of mixer elements, for example vortex inducers 404, spacer 406, mixer array 408, or any combination thereof, come together to form a tortuous fluid pathway which encourages mixing and exposes the fluid to shear stress.

In some cases, the mixer design can encourage mixing by limiting the fluid domain (e.g, where the fluid can reside). By narrowing the fluid domain, the mixer imparts additional shear stress on the fluid to increase the gel yield stress. The application of shear stress through mixing can increase the viscosity of the fluid (e.g., the multi-component fluid). The viscosity of the multi-component fluid can range from about 5 centipoise (cP) to about 1,000,000 cP (1 cP=1 mPa*s) as discussed above.

The mixer 400 can comprise multiple stages. The mixer 400 can comprise an array of stages 408. The mixer 400 can comprise an array 408 of 12 stages. The mixer 400 can comprise an array 408 of less than 12 stages. The mixer 400 can comprise an array of 1, 2, 3, 4, 5, 6, 7, 8, 9, 10, 11, 12, 13, 14, 15, 16, 17, 18, 19, 20, or more stages.

The diameter of the mixer stages can be between about 2 mm and about 20 mm. The diameter of the mixer stages can be between about 2 mm to about 4 mm, about 2 mm to about 6 mm, about 2 mm to about 8 mm, about 2 mm to about 10 mm, about 2 mm to about 12 mm, about 2 mm to about 14 mm, about 2 mm to about 16 mm, about 2 mm to about 18 mm, about 2 mm to about 20 mm, about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 10 mm to about 12 mm, about 10 mm to about 14 mm, about 10 mm to about 16 mm, about 10 mm to about 18 mm, about 10 mm to about 20 mm, about 12 mm to about 14 mm, about 12 mm to about 16 mm, about 12 mm to about 18 mm, about 12 mm to about 20 mm, about 14 mm to about 16 mm, about 14 mm to about 18 mm, about 14 mm to about 20 mm, about 16 mm to about 18 mm, about 16 mm to about 20 mm, or between about 18 mm to about 20 mm. The diameter can be less than about 2 mm, less than about 4 mm, less than about 6 mm, less than about 8 mm, less than about 10 mm, less than about 12 mm, less than about 14 mm, less than about 16 mm, less than about 18 mm, or less than about 20 mm. The diameter can be greater than about 2 mm, greater than about 4 mm, greater than about 6 mm, greater than about 8 mm, greater than about 10 mm, greater than about 12 mm, greater than about 14 mm, greater than about 16 mm, greater than about 18 mm, or greater than about 20 mm.

In some cases, the diameter of the mixer stages can be between about 1 mm and about 5 mm. The diameter of the mixer stages can be between about 1 mm to about 2 mm, about 1 mm to about 3 mm, about 1 mm to about 4 mm, about 1 mm to about 5 mm, about 2 mm to about 3 mm, about 2 mm to about 4 mm, about 2 mm to about 5 mm, about 3 mm to about 4 mm, about 3 mm to about 5 mm, or between about 4 mm to about 5 mm. The diameter can be less than 1 mm, less than 2 mm, less than 3 mm, less than 4 mm, or less than 5 mm. The diameter can be greater than 1 mm, greater than 2 mm, greater than 3 mm, greater than 4 mm, or greater than 5 mm. In some cases, the diameter is about 3 mm.

The total length of all of the mixer stages can be between about 20 mm and about 60 mm. The length can be between about 20 mm to about 25 mm, about 20 mm to about 30 mm, about 20 mm to about 35 mm, about 20 mm to about 40 mm, about 20 mm to about 45 mm, about 20 mm to about 50 mm, about 20 mm to about 55 mm, about 20 mm to about 60 mm, about 25 mm to about 30 mm, about 25 mm to about 35 mm, about 25 mm to about 40 mm, about 25 mm to about 45 mm, about 25 mm to about 50 mm, about 25 mm to about 55 mm, about 25 mm to about 60 mm, about 30 mm to about 35 mm, about 30 mm to about 40 mm, about 30 mm to about 45 mm, about 30 mm to about 50 mm, about 30 mm to about 55 mm, about 30 mm to about 60 mm, about 35 mm to about 40 mm, about 35 mm to about 45 mm, about 35 mm to about 50 mm, about 35 mm to about 55 mm, about 35 mm to about 60 mm, about 40 mm to about 45 mm, about 40 mm to about 50 mm, about 40 mm to about 55 mm, about 40 mm to about 60 mm, about 45 mm to about 50 mm, about 45 mm to about 55 mm, about 45 mm to about 60 mm, about 50 mm to about 55 mm, about 50 mm to about 60 mm, or between about 55 mm to about 60 mm. The length can be at least about 20 mm, at least about 25 mm, at least about 30 mm, at least about 35 mm, at least about 40 mm, at least about 45 mm, at least about 50 mm, at least about 55 mm, at least about 60 mm. The length can be at most about 20 mm, at most about 25 mm, at most about 30 mm, at most about 35 mm, at most about 40 mm, at most about 45 mm, at most about 50 mm, at most about 55 mm, at most about 60 mm. The length can be 47.5 mm.

The approximate volume of all of the mixer stages combined can be between about 10 mm³ and about 1000 mm³. The approximate volume of the mixer stages can be between about 10 mm³ to about 200 mm³, about 10 mm³ to about 400 mm³, about 10 mm³ to about 600 mm³, about 10 mm³ to about 800 mm³, about 10 mm³ to about 1000 mm³, about 200 mm³ to about 400 mm³, about 200 mm³ to about 600 mm³, about 200 mm³ to about 800 mm³, about 200 mm³ to about 1000 mm³, about 400 mm³ to about 600 mm³, about 400 mm³ to about 800 mm³, about 400 mm³ to about 1000 mm³, about 600 mm³ to about 800 mm³, about 600 mm³ to about 1000 mm³, or between about 800 mm³ to about 1000 mm³. The approximate volume of the mixer stages can be less than 10 mm³. The approximate volume of the mixer stages can be greater than 1000 mm³.

In some cases, the approximate volume of all of the mixer stages combined can be between about 200 mm³ to about 300 mm³. In some cases, the approximate volume of the all of mixer stages combined can be between about 200 mm³ to about 220 mm³, about 200 mm³ to about 240 mm³, about 200 mm³ to about 260 mm³, about 200 mm³ to about 280 mm³, about 200 mm³ to about 300 mm³, about 220 mm³ to about 240 mm³, about 220 mm³ to about 260 mm³, about 220 mm³ to about 280 mm³, about 220 mm³ to about 300 mm³, about 240 mm³ to about 260 mm³, about 240 mm³ to about 280 mm³, about 240 mm³ to about 300 mm³, about 260 mm³ to about 280 mm³, about 260 mm³ to about 300 mm³, or between about 280 mm³ to about 300 mm³. The volume can be less than about 200 mm³, less than about 210 mm³, less than about 220 mm³, less than about 230 mm³, less than about 240 mm³, less than about 250 mm³, less than about 260 mm³, less than about 270 mm³, less than about 280 mm³, less than about 290 mm³, or less than about 300 mm³. The volume can be greater than about 200 mm³, greater than about 210 mm³, greater than about 220 mm³, greater than about 230 mm³, greater than about 240 mm³, greater than about 250 mm³, greater than about 260 mm³, greater than about 270 mm³, greater than about 280 mm³, greater than about 290 mm³, or greater than about 300 mm³. In some cases, the volume is 248.2 mm³.

The diameter of the mixer body 412 can be between about 3 mm and about 25 mm. The diameter of the mixer body 412 can be between about 3 mm to 5 mm, about 3 mm to 10 mm, about 3 mm to 15 mm, about 3 mm to 20 mm, about 3 mm to 25 mm, about 5 mm to 10 mm, about 5 mm to 15 mm, about 5 mm to 20 mm, about 5 mm to 25 mm, about 10 mm to 15 mm, about 10 mm to 20 mm, about 10 mm to 25 mm, about 15 mm to 20 mm, about 15 mm to 25 mm, or between about 20 mm to 25 mm. The diameter can be at least about 3 mm, at least about 5 mm, at least about 10 mm, at least about 15 mm, at least about 20 mm, at least about 25 mm. The diameter can be at most about 3 mm, at most about 5 mm, at most about 10 mm, at most about 15 mm, at most about 20 mm, at most about 25 mm.

In some cases, the lumen of mixer body 412 has a volume, which will be occupied by a mixer and/or mixer elements and fluid. Mixer elements of a greater volume may result in less loss volume (e.g. reduced space occupied by fluid).

The approximate volume of the mixer body 412 overlaying the mixer array 408 can be between about 200 mm³ and about 800 mm³. The approximate volume of the mixer body 412 overlaying the mixer array 408 can be between about 200 mm³ to about 400 mm³, about 200 mm³ to about 600 mm³, about 200 mm³ to about 800 mm³, about 400 mm³ to about 600 mm³, about 400 mm³ to about 800 mm³, or between about 600 mm³ to about 800 mm³. The approximate volume of the mixer body 412 overlaying the mixer array 408 can be less than about 200 mm³, less than about 300 mm³, less than about 400 mm³, less than about 500 mm³, less than about 600 mm³, less than about 700 mm³, or less than about 800 mm³. The approximate volume of the mixer body 412 overlaying the mixer array 408 can be greater than about 200 mm³, greater than about 300 mm³, greater than about 400 mm³, greater than about 500 mm³, greater than about 600 mm³, greater than about 700 mm³, or greater than about 800 mm³. The approximate volume of the mixer body 412 overlaying the mixer array 408 can be less than 200 mm³. The approximate volume of the mixer body 412 overlaying the mixer array 408 can be greater than 800 mm³. In some cases, the volume of mixer body 412 is about 551.3 mm³.

The difference in volume can comprise the fluid domain for the multi-component fluid to move through as it proceeds from hypotubes 410 to the nozzle tip 402. The multi-component fluid can be squeezed through the annular gap that results between the mixer array 408 and the mixer body 412.

In some cases, the mixer stages are semi-circular. In some embodiments, the mixer pieces comprise a spacer, a blade, a fin, or a channel. The semi-circular design can comprise more area and/or bulk than the other designs of mixer pieces. In some cases, the semi-circular design is more compact. The semi-circular design can result in lower lost volume than an alternate design.

In some cases, the distal end of the mixer 400 can comprise one or more vortex inducers 404. The one or more vortex inducers 404 can impose shear stress on the multi-component fluid to generate a high yield stress (e.g. between about 200 Pa and about 700 Pa) of the resulting gel as discussed above. Shear stress can be derived using a computational model. The computational model can use artificial intelligence or machine learning computing. In some cases, the vortex inducers can also impose rotation or swirl (e.g. vorticity) on the multi-component liquid.

In some cases, the mixer 400 can comprise one or more spacers 406. The spacer 406 can be located between vortex inducers 404. In some cases, with reference to FIGS. 5A and 5B, the distal end of a mixer 400 comprises a vortex inducer 404, a spacer 406, and a second vortex inducer 404. There may be multiple iterations of vortex inducer 404, spacer 406, and vortex inducer 404 to impart further shear stress on the multi-component fluid. In some cases, there may be multiple vortex inducers in a row without a spacer in between. In some cases, one can regulate the shear stress exerted on the multi-component fluid, and thus the yield stress of the resulting gel, by regulating the amount and order of the vortex inducers 404.

In some cases, the mixer 400 does not comprise one or more vortex inducers. In some cases, the mixer 104 does not comprise one or more spacers. The mixer 400 can be fluidly coupled to the nozzle outlet and/or the outlet of device.

A delivery device as described herein may comprise a nozzle disposed distal to the mixer 400. In some cases, there is no clear division of the nozzle versus the mixer. In some cases, the vortex inducers and spacers can be considered to be in the mixer or the nozzle. The nozzle may comprise a nozzle inlet, a nozzle body, and a nozzle outlet. The nozzle can receive the multi-component fluid from the mixer and deliver the multi-component fluid through the nozzle outlet to a location external to the device. The application of shear stress through mixing can alter the viscosity. The viscosity of the multi-component fluid can range from about 5 centipoise (cP) to about 1,000,000 cP (1 cP=1 mPa*s) as discussed above.

In some cases, one or more of the nozzle inlet, nozzle body, or nozzle outlet can comprise one or more vortex inducers. The one or more vortex inducers can impose shear stress on the multi-component fluid to generate a high yield stress (e.g. between about 200 Pa and about 700 Pa) of the resulting gel as discussed above. Shear stress can be derived using a computational model.

In some cases, one or more of the nozzle inlet, nozzle body, or nozzle outlet can comprise one or more spacers. The spacer can be located between vortex inducers. In some cases, the nozzle comprises a nozzle inlet comprising a vortex inducer, a nozzle body comprising a spacer, and a nozzle outlet comprising a second vortex inducer. There may be multiple iterations of vortex inducer, spacer, vortex inducer to impart further shear stress on the multi-component fluid. In some cases, there may be multiple vortex inducers in a row without a spacer in between. In some cases, the entirety of the nozzle comprises a vortex inducer without a spacer.

The nozzle tip 402 comprises a nozzle outlet. The nozzle tip 402 releases the completed multi-component fluid. In some cases, when the viscosity is low, the multi-component fluid is liquid and can exit the nozzle tip 402 quickly. The multi-component fluid can exit the nozzle tip 402 as a stream or liquid or be dispersed as particles. In some cases, when the viscosity is high, the multi-component fluid comprises a gel-like consistency (e.g., a hydrogel) and can exit the nozzle tip 402 slowly.

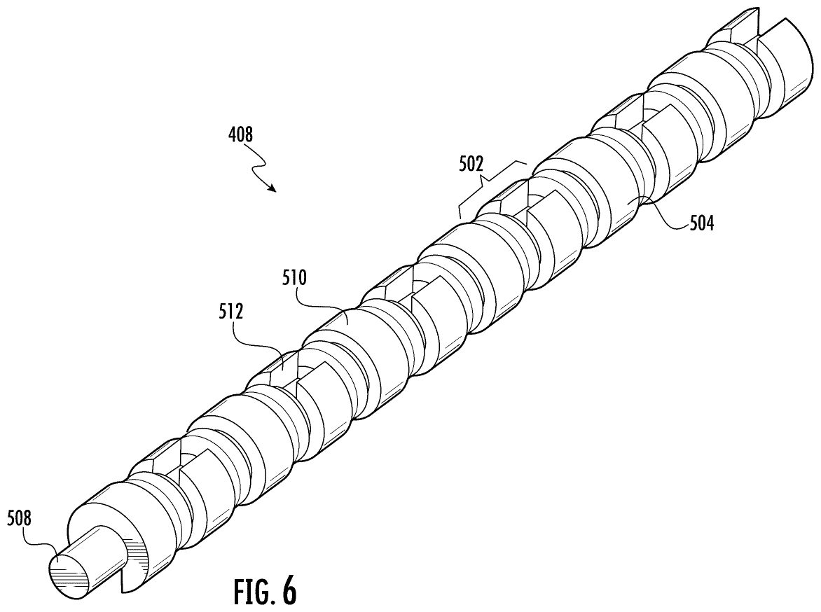

FIG. 6 shows an expanded perspective view of an exemplary embodiment of mixers in the mixing element and/or a mixer referred to in FIGS. 5A and 5B. FIG. 6 is a focused version of mixer array 408 showing individual mixer stages 502. The mixer array shown in FIG. 6 has 12 mixer stages 502, but the mixer array can have more or less stages, as discussed above. The mixer array 408 can comprise an array of 1, 2, 3, 4, 5, 6, 7, 8, 9, 10, 11, 12, 13, 14, 15, 16, 17, 18, 19, 20, or more stages 502. As shown, the mixer stage 502 can be configured as a band or ring 504 extending from or connected to a central shaft 508. Each of the rings or bands 504 can have a round outer surface 510 having cutaway slot 512, as shown in FIG. 6. In an embodiment, the rings 510 can be staggered along the shaft 508 such that the cutaway slots 512 of two adjacent mixer stages 502 are not aligned, as illustrated.

The diameter of the mixer stages 502 can be between about 2 mm and about 20 mm. The diameter of the mixer stages can be between about 2 mm to about 4 mm, about 2 mm to about 6 mm, about 2 mm to about 8 mm, about 2 mm to about 10 mm, about 2 mm to about 12 mm, about 2 mm to about 14 mm, about 2 mm to about 16 mm, about 2 mm to about 18 mm, about 2 mm to about 20 mm, about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 10 mm to about 12 mm, about 10 mm to about 14 mm, about 10 mm to about 16 mm, about 10 mm to about 18 mm, about 10 mm to about 20 mm, about 12 mm to about 14 mm, about 12 mm to about 16 mm, about 12 mm to about 18 mm, about 12 mm to about 20 mm, about 14 mm to about 16 mm, about 14 mm to about 18 mm, about 14 mm to about 20 mm, about 16 mm to about 18 mm, about 16 mm to about 20 mm, or between about 18 mm to about 20 mm. The diameter can be less than about 2 mm, less than about 4 mm, less than about 6 mm, less than about 8 mm, less than about 10 mm, less than about 12 mm, less than about 14 mm, less than about 16 mm, less than about 18 mm, or less than about 20 mm. The diameter can be greater than about 2 mm, greater than about 4 mm, greater than about 6 mm, greater than about 8 mm, greater than about 10 mm, greater than about 12 mm, greater than about 14 mm, greater than about 16 mm, greater than about 18 mm, or greater than about 20 mm.

The total length of all of the mixer stages can be between about 20 mm and about 60 mm. The length can be between about 20 mm to about 25 mm, about 20 mm to about 30 mm, about 20 mm to about 35 mm, about 20 mm to about 40 mm, about 20 mm to about 45 mm, about 20 mm to about 50 mm, about 20 mm to about 55 mm, about 20 mm to about 60 mm, about 25 mm to about 30 mm, about 25 mm to about 35 mm, about 25 mm to about 40 mm, about 25 mm to about 45 mm, about 25 mm to about 50 mm, about 25 mm to about 55 mm, about 25 mm to about 60 mm, about 30 mm to about 35 mm, about 30 mm to about 40 mm, about 30 mm to about 45 mm, about 30 mm to about 50 mm, about 30 mm to about 55 mm, about 30 mm to about 60 mm, about 35 mm to about 40 mm, about 35 mm to about 45 mm, about 35 mm to about 50 mm, about 35 mm to about 55 mm, about 35 mm to about 60 mm, about 40 mm to about 45 mm, about 40 mm to about 50 mm, about 40 mm to about 55 mm, about 40 mm to about 60 mm, about 45 mm to about 50 mm, about 45 mm to about 55 mm, about 45 mm to about 60 mm, about 50 mm to about 55 mm, about 50 mm to about 60 mm, or between about 55 mm to about 60 mm. The length can be at least about 20 mm, at least about 25 mm, at least about 30 mm, at least about 35 mm, at least about 40 mm, at least about 45 mm, at least about 50 mm, at least about 55 mm, at least about 60 mm. The length can be at most about 20 mm, at most about 25 mm, at most about 30 mm, at most about 35 mm, at most about 40 mm, at most about 45 mm, at most about 50 mm, at most about 55 mm, at most about 60 mm. The length can be 47.5 mm.

The approximate volume of all of the mixer stages combined can be between about 10 mm³ and about 1000 mm³. The approximate volume of the mixer stages can be between about 10 mm³ to about 200 mm³, about 10 mm³ to about 400 mm³, about 10 mm³ to about 600 mm³, about 10 mm³ to about 800 mm³, about 10 mm³ to about 1000 mm³, about 200 mm³ to about 400 mm³, about 200 mm³ to about 600 mm³, about 200 mm³ to about 800 mm³, about 200 mm³ to about 1000 mm³, about 400 mm³ to about 600 mm³, about 400 mm³ to about 800 mm³, about 400 mm³ to about 1000 mm³, about 600 mm³ to about 800 mm³, about 600 mm³ to about 1000 mm³, or between about 800 mm³ to about 1000 mm³. The approximate volume of the mixer stages can be less than 10 mm³. The approximate volume of the mixer stages can be greater than 1000 mm³.

In some cases, the approximate volume of all of the mixer stages combined can be between about 200 mm³ to about 300 mm³. In some cases, the approximate volume of the all of mixer stages combined can be between about 200 mm³ to about 220 mm³, about 200 mm³ to about 240 mm³, about 200 mm³ to about 260 mm³, about 200 mm³ to about 280 mm³, about 200 mm³ to about 300 mm³, about 220 mm³ to about 240 mm³, about 220 mm³ to about 260 mm³, about 220 mm³ to about 280 mm³, about 220 mm³ to about 300 mm³, about 240 mm³ to about 260 mm³, about 240 mm³ to about 280 mm³, about 240 mm³ to about 300 mm³, about 260 mm³ to about 280 mm³, about 260 mm³ to about 300 mm³, or between about 280 mm³ to about 300 mm³. The volume can be less than about 200 mm³, less than about 210 mm³, less than about 220 mm³, less than about 230 mm³, less than about 240 mm³, less than about 250 mm³, less than about 260 mm³, less than about 270 mm³, less than about 280 mm³, less than about 290 mm³, or less than about 300 mm³. The volume can be greater than about 200 mm³, greater than about 210 mm³, greater than about 220 mm³, greater than about 230 mm³, greater than about 240 mm³, greater than about 250 mm³, greater than about 260 mm³, greater than about 270 mm³, greater than about 280 mm³, greater than about 290 mm³, or greater than about 300 mm³. In some cases, the volume is 248.2 mm³.

In some cases, the mixer stages 502 are semi-circular. In some embodiments, the mixer pieces comprise a spacer, a blade, a fin, or a channel. The semi-circular design can comprise more area and/or bulk than the other designs of mixer pieces. In some cases, the semi-circular design is more compact. The semi-circular design can result in lower lost volume than an alternate design.

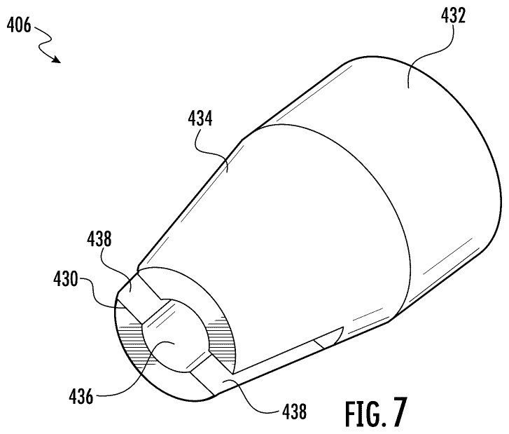

FIG. 7 shows an expanded perspective view of an exemplary embodiment of a spacer 406 in the mixing element and/or mixer referred to in FIGS. 5A and 5B. There can be one or more spacers 406 in the mixer and/or nozzle. The spacer 406 can be located distal to or proximal to a vortex inducer. The spacer 406 can be located between vortex inducers.

The spacer 406 can comprise a cylindrical portion 432 and a narrowing nose portion 434. The distal end 430 of the nose portion 434 can extend into a central opening 436 and comprise a circle or an ellipse. The spacer 406 can comprise incisions 438 in the nose portion 434 to improve attachment to a vortex inducer. The diameter of the cylindrical portion 432 can be between about 2 mm to about 20 mm. The diameter of the cylindrical portion can be between about 2 mm to about 4 mm, about 2 mm to about 6 mm, about 2 mm to about 8 mm, about 2 mm to about 10 mm, about 2 mm to about 12 mm, about 2 mm to about 14 mm, about 2 mm to about 16 mm, about 2 mm to about 18 mm, about 2 mm to about 20 mm, about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 10 mm to about 12 mm, about 10 mm to about 14 mm, about 10 mm to about 16 mm, about 10 mm to about 18 mm, about 10 mm to about 20 mm, about 12 mm to about 14 mm, about 12 mm to about 16 mm, about 12 mm to about 18 mm, about 12 mm to about 20 mm, about 14 mm to about 16 mm, about 14 mm to about 18 mm, about 14 mm to about 20 mm, about 16 mm to about 18 mm, about 16 mm to about 20 mm, or between about 18 mm to about 20 mm. The diameter can be less than about 2 mm, less than about 4 mm, less than about 6 mm, less than about 8 mm, less than about 10 mm, less than about 12 mm, less than about 14 mm, less than about 16 mm, less than about 18 mm, or less than about 20 mm. The diameter can be greater than about 2 mm, greater than about 4 mm, greater than about 6 mm, greater than about 8 mm, greater than about 10 mm, greater than about 12 mm, greater than about 14 mm, greater than about 16 mm, greater than about 18 mm, or greater than about 20 mm.

The diameter of the distal end 430 of the nose portion 434 can be between about 2 mm to about 10 mm. The diameter of the distal end of the nose portion can be between about 2 mm to about 4 mm, about 2 mm to about 6 mm, about 2 mm to about 8 mm, about 2 mm to about 10 mm, about 4 mm to about 6 mm, about 4 mm to about 8 mm, about 4 mm to about 10 mm, about 6 mm to about 8 mm, about 6 mm to about 10 mm, and finally, or between about 8 mm to about 10 mm.

The length of the spacer 406 can be between about 2 mm to about 20 mm. The length of the spacer 406 can be between about 2 mm to about 4 mm, about 2 mm to about 6 mm, about 2 mm to about 8 mm, about 2 mm to about 10 mm, about 2 mm to about 12 mm, about 2 mm to about 14 mm, about 2 mm to about 16 mm, about 2 mm to about 18 mm, about 2 mm to about 20 mm, about 4 mm to about 6 mm, about 4 mm to about 8 mm, about 4 mm to about 10 mm, about 4 mm to about 12 mm, about 4 mm to about 14 mm, about 4 mm to about 16 mm, about 4 mm to about 18 mm, about 4 mm to about 20 mm, about 6 mm to about 8 mm, about 6 mm to about 10 mm, about 6 mm to about 12 mm, about 6 mm to about 14 mm, about 6 mm to about 16 mm, about 6 mm to about 18 mm, about 6 mm to about 20 mm, about 8 mm to about 10 mm, about 8 mm to about 12 mm, about 8 mm to about 14 mm, about 8 mm to about 16 mm, about 8 mm to about 18 mm, about 8 mm to about 20 mm, about 10 mm to about 12 mm, about 10 mm to about 14 mm, about 10 mm to about 16 mm, about 10 mm to about 18 mm, about 10 mm to about 20 mm, about 12 mm to about 14 mm, about 12 mm to about 16 mm, about 12 mm to about 18 mm, about 12 mm to about 20 mm, about 14 mm to about 16 mm, about 14 mm to about 18 mm, about 14 mm to about 20 mm, about 16 mm to about 18 mm, about 16 mm to about 20 mm, or between about 18 mm to about 20 mm. The length can be less than about 2 mm, less than about 4 mm, less than about 6 mm, less than about 8 mm, less than about 10 mm, less than about 12 mm, less than about 14 mm, less than about 16 mm, less than about 18 mm, or less than about 20 mm. The length can be greater than about 2 mm, greater than about 4 mm, greater than about 6 mm, greater than about 8 mm, greater than about 10 mm, greater than about 12 mm, greater than about 14 mm, greater than about 16 mm, greater than about 18 mm, or greater than about 20 mm.

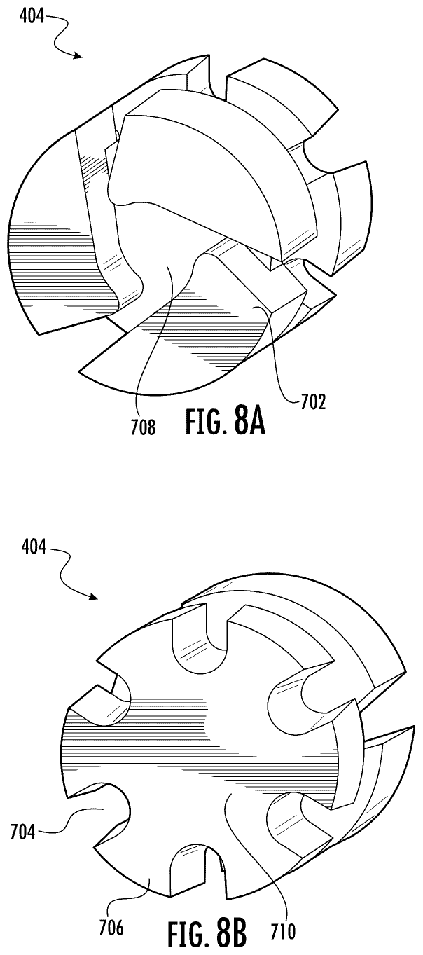

FIGS. 8A and 8B depict perspective proximate and perspective distal views, respectively, of a vortex inducer 404 in the mixing element referred to in FIGS. 5A and 5B. Vortex inducer 404 can comprise channel guide 710, blades or vanes 702, lateral fluid paths 704, channel guide barriers 706, and cylindrical swirl-inducing connector region 708.

The channel guide 710 on the other side can have one or more lateral fluid paths 704, formed by solid barriers between the channels 706. The fluids being mixed are conveyed from the lateral fluid paths 704 to a central channel. In some cases, the fluids are conveyed via a path that induces swirl. Lateral fluid paths 704 can comprise an alternating series of channels. In some embodiments, the alternating series of channels and channel guide barriers 706 form a grouping of lateral flow pathways. The fluids can then be conveyed through a set of channels perpendicular to the long axis of the mixer into the region which induces swirl 708.

The vortex inducer 404 can comprise one or more blades 702 on one side and a channel guide 710 on the other side. In some cases, the maximum diameter of the blades 702 on one side and channel guide 710 on the other side are the same. In some cases, the maximum diameter of the blades 702 on one side and channel guide 710 on the other side are different.

In some cases, the central channel can be a spacer, for example the spacer of FIG. 7. In some cases, the fluids are conveyed directly to a nozzle. The channel guide 710 can have 1, 2, 3, 4, 5, 6, 7, 8, 9, 10 or more lateral fluid paths. In some cases, there are 6 lateral fluid paths 706 on the channel guide 710. The lateral fluid paths 704 between the channel guide barriers 706 can be semi-circular or semi-elliptical cylinders. In some cases, there is a channel guide 710 on both sides of the vortex inducer. In some cases, the number of lateral fluid paths 704 on each side can vary.

There can be 1, 2, 3, 4, 5, 6, 7, 8, 9, 10 or more blades or vanes 702 on one side. In some cases, there are three blades or vanes 702 on one side. The blades or vanes 702 can be at 60 degree angles with respect to each other. In some cases, the different in angles of the blades or vanes 702 with respect to each other can be calculated as 180 degrees divided by the number of blades or vanes 702. In some cases, there are blades or vanes 702 on both sides of the vortex inducer. In some cases, the number of blades or vanes on each side can vary.

In some cases, there is a cylinder 708 disposed between the blades or vanes 702 on one side of the vortex inducer 404 and the channel guide 710 on the other side. The cylinder 708 can connect the two sides.

The length of the cylinder 708 can be between 2 mm to about 10 mm. The length of the cylinder 708 can be between about 2 mm to about 4 mm, about 2 mm to about 6 mm, about 2 mm to about 8 mm, about 2 mm to about 10 mm, about 4 mm to about 6 mm, about 4 mm to about 8 mm, about 4 mm to about 10 mm, about 6 mm to about 8 mm, about 6 mm to about 10 mm, and finally, or between about 8 mm to about 10 mm. In some cases, the length of the cylinder 708 can be at least about 2 mm, at least about 4 mm, at least about 6 mm, at least about 8 mm, at least about 10 mm. In some cases, the length of the cylinder 708 can be at most about 2 mm, at most about 4 mm, at most about 6 mm, at most about 8 mm, at most about 10 mm.

The diameter of the cylinder 708 can be between about 2 mm to about 18 mm. The diameter of the cylinder 708 can be between about 2 mm to about 6 mm, about 2 mm to about 10 mm, about 2 mm to about 14 mm, about 2 mm to about 18 mm, about 6 mm to about 10 mm, about 6 mm to about 14 mm, about 6 mm to about 18 mm, and about 10 mm to about 14 mm, or between about 10 mm to about 18 mm. The diameter can be less than 2 mm. The diameter can be greater than 18 mm.

The vortex inducer 404 can serve multiple functions near the distal end of a device as described herein. The vortex inducer 404 can induce swirling of the fluid (e.g. induces a vortex, vorticity). The vortex inducer 404 can also impart shear stress on the multi-component fluid as it flows by, thereby increasing the gel yield stress. In some cases, multiple vortex inducers 404 can be used in one device to increase these effects.

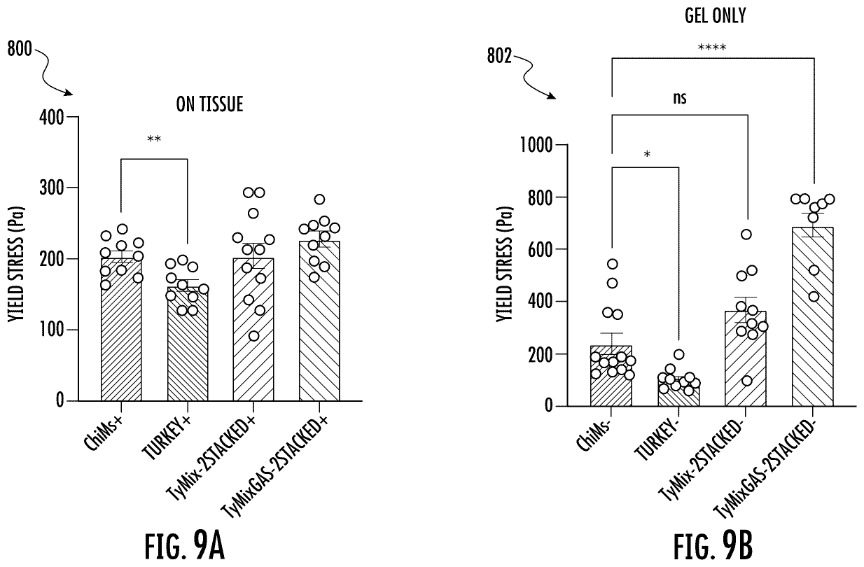

FIG. 9A illustrates a graph of yield stress of a multi-component fluid when embodiments of devices described herein are used on tissue. FIG. 9B illustrates a graph of yield stress of a multi-component fluid when embodiments of devices described herein are used. ChiMS is a standard syringe dispensing through a gas-assisted nozzle, Turkey is device 300, TyMix-2Stacked is device 100, and TyMixGAS-2Stacked is device 200 described herein. The hydrogel yield stress of a multi-component fluid can be measured on different surfaces (e.g. with and without tissue). FIG. 9A shows a graph of yield stress measured on a tissue surface and FIG. 9B depicts a graph of yield stress measured on a metal surface. In some cases, yield stress for a hydrogel adhered to tissue can be about 200 Pa for all four dispensing methods. When yield stress was measured without tissue, both airless device 100 and gas-assisted device 200 increased yield stress to 372 Pa and 693 Pa, respectively. Device 300 decreased the yield stress by 60 Pa and the regular syringe dispensing method increased by about 40 Pa. The experiment is described in further detail in Example 6 below.

### Methods

Described herein are methods for delivering a multi-component fluid using the devices described herein. In some cases, the methods for delivering a multi-component fluid comprises providing a delivery device as described herein. The delivery device can comprise a housing. The housing can have a first lumen and a second lumen. The first lumen can be configured to receive a first component of the multi-component fluid. The second lumen can be configured to receive a second component of the multi-component fluid. A mixer can be coupled to the housing. The mixer can comprise one or more sub-mixers (e.g. mixer stages). A proximal end of the mixer may be in fluid communication with the first lumen and with the second lumen. The proximal end of the mixer can receive the first component and the second component within the mixer. The mixer can mix the first component and the second component to form the multi-component fluid. The housing can comprise a nozzle couple to the mixer. The nozzle can comprise one or more vortex inducers and a nozzle outlet. The nozzle can receive the multi-component fluid from the mixer and deliver the multi-component fluid from the nozzle outlet.

The method for delivering a multi-component fluid can comprise delivering the first component and the second component through the first lumen and second lumen respectively. The first component and the second component can mix via the mixer to form the multi-component fluid. The method can comprise delivering the multi-component fluid through one or more vortex inducers in the nozzle. In some cases, the method comprises delivering the multi-component fluid through the nozzle outlet.

Provided herein are methods of use for treatment of a wound. With reference to the device 100 of FIG. 1A comprising a housing with a dual-barreled cartridge holder 110, a user can insert two fluid compounds, one in each of the cartridges. One or more of the fluid compounds can be a buffer. One or more of the fluid compounds can comprise an extracellular matrix (ECM). The user can insert the fluid-filled cartridges into the dual-barreled cartridge holder 110. In some cases, the cartridges can be in fluid communication with containers holding one or more types of fluid compounds. There can be two containers, one for a buffer and one for an ECM.

The user can place his thumb on the thumb grip 120 and his index and middle fingers on the two extensions 122A, 122B of finger grip 112. The user can place the delivery device 100 near a wound, abrasion, cut, surgical site, or other condition that can benefit from separation of tissues or anatomic structures without undesirable attachment of internal components. While holding the device 100 near the intended area of use, the user can slowly press down on the thumb grip 120. In some cases, ratcheted dispenser 114 can instead be a piston, a plunger, a pump, a multi-stage ratcheted button, a different actuator, or any combination thereof. The distal end of the ratcheted dispenser 114 is connected to the proximal end of the housing and the proximal ends of the dual-barreled cartridge holder 110. Pushing down on the thumb grip 120 can cause the fluids in the cartridges to move into the arthroscopic extension 106, thereby imparting shear stress on the fluids due to the narrowing space for the fluids to occupy. The fluids can remain separate until this point. The fluids can be combined at this point.

In some cases, the space narrows from between about 3 mm to about 25 mm in the cartridges to between about 0.1 mm to about 1 mm in the arthroscopic extension. In some cases, the space narrows from between about 3 mm to about 5 mm, about 3 mm to about 10 mm, about 3 mm to about 15 mm, about 3 mm to about 20 mm, about 3 mm to about 25 mm, about 5 mm to about 10 mm, about 5 mm to about 15 mm, about 5 mm to about 20 mm, about 5 mm to about 25 mm, about 10 mm to about 15 mm, about 10 mm to about 20 mm, about 10 mm to about 25 mm, about 15 mm to about 20 mm, about 15 mm to about 25 mm, or between about 20 mm to about 25 mm. In some cases, the space narrows from less than about 3 mm, less than bout 5 mm, less than about 10 mm, less than about 15 mm, less than about 20 mm, or less than about 25 mm. In some cases, the space narrows from greater than about 3 mm, greater than bout 5 mm, greater than about 10 mm, greater than about 15 mm, greater than about 20 mm, or greater than about 25 mm. In some cases, the space narrows from about 6.85 mm or about 12 mm.

In some embodiments, the space narrows in the arthroscopic extension to between about 0.1 mm to about 0.2 mm, about 0.1 mm to about 0.4 mm, about 0.1 mm to about 0.6 mm, about 0.1 mm to about 0.8 mm, about 0.1 mm to about 1 mm, about 0.2 mm to about 0.4 mm, about 0.2 mm to about 0.6 mm, about 0.2 mm to about 0.8 mm, about 0.2 mm to about 1 mm, about 0.4 mm to about 0.6 mm, about 0.4 mm to about 0.8 mm, about 0.4 mm to about 1 mm, about 0.6 mm to about 0.8 mm, about 0.6 mm to about 1 mm, or between about 0.8 mm to about 1 mm. In some embodiments, the space narrows in the arthroscopic extension to less than about 0.1 mm, less than about 0.2 mm, less than about 0.4 mm, less than about 0.6 mm, less than about 0.8 mm, or less than about 1 mm. In some embodiments, the space narrows in the arthroscopic extension to greater than about 0.1 mm, greater than about 0.2 mm, greater than about 0.4 mm, greater than about 0.6 mm, greater than about 0.8 mm, or greater than about 1 mm. In some cases, the space narrows to between about 0.3 mm and about 0.6 mm. In some cases, the space narrows to between about 0.3 mm to about 0.4 mm, about 0.3 mm to about 0.5 mm, about 0.3 mm to about 0.6 mm, about 0.4 mm to about 0.5 mm, about 0.4 mm to about 0.6 mm, about 0.5 mm to about 0.6 mm. In some cases, the space narrows to about 0.59 mm or about 0.35 mm.

After continued pushing or actuating of the thumb grip 120 and/or ratcheted dispenser 114, the fluids can pass from the arthroscopic extension 106 to the mixer 104. The mixer further decreases the fluid domain available to the multi-component fluid (e.g, the space inside the device 100 for fluid to flow). The fluids can be mixed in the mixer. The fluids can then be pushed out of the device, mixer, and nozzle by additional pushing or actuation of the thumb grip 120 and/or ratcheted dispenser 114.

In some cases, while keeping the device near a wound, abrasion, cut, surgical site, or other condition, the user can push out the multi-component fluid onto the injured tissue. In some embodiments, the multi-component fluid is viscous and comes out as a gel, for example, a hydrogel. In some cases, the multi-component fluid is not viscous and can come out as a liquid or a spray.

In some cases, the device 200 of FIG. 3 can be used in a similar way. In some cases, device 200 can function with a gas assist. Device 200 can function without a gas assist. The user can attach a gas tube to the gas assist luer port 218. The gas can comprise a compressed gas. In some embodiments, the compressed gas may comprise oxygen, carbon dioxide, Nitrogen, helium, atmospheric air, argon, neon, xenon, krypton, radon, acetylene, butane, ethylene, hydrogen, methylamine, vinyl chloride, nitrogen oxides, halogen gases (e.g., chlorine, fluorine), acetylene,1,3-butadiene, methyl acetylene, tetrafluoroethylene, vinyl fluoride, or combinations thereof. In some cases, the gas can help to propel the fluid throughout the fluid path to the nozzle by adding pressure. When the user is ready to apply the multi-component fluid, the user can activate the gas (e.g. turn on a gas supplying machine, squeeze a container with compressed gas, etc.) to speed up the mixing and pushing processes. In some cases, a steady flow of gas can be supplied through the gas-assist luer port. In some cases, the amount of compressed gas can be regulated by the device that supplies the gas.

In some cases, the device 300 of FIGS. 4A and 4B can be used in a similar way. The device 300 can use a carbon dioxide gas assist through a gas tube.

### Exemplary Method

An example of using a device disclosed herein may be provided. During a surgical procedure in the abdomen or pelvis, access sites may be created, either by minimally invasive techniques including laparoscopic and robotic approaches or by traditional opened surgeries such as laparotomies. The surgeon inserts the appropriated instruments to perform the procedure. At the conclusion of the procedure all surgical tools and instruments may be withdrawn. At this point, the nozzle end of the delivery system described here could be inserted through the access site and guided to the site of the procedure by use of the steering mechanism. Upon pressing the button to spray, the dispenser will depress the plungers such that their contents will move through the tube where they will enter the mixer. In some embodiments, the dispenser will depress the plungers such that their contents will move through the tube where they will enter the mixer by way of a constant force spring. At the distal end of the mixer, they will exit through the nozzle with air assist to form small droplets which will gel on contact with the warm tissue. The surgeon will continue until the surfaces of the organs and abdominal wall may be coated. The delivery device can be withdrawn and surgical access sites closed.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. The term “about” or “approximately” may mean within an acceptable error range for the particular value, which will depend in part on how the value is measured or determined, e.g., the limitations of the measurement system. For example, the terms “approximately”, “about”, and “substantially” may refer to an amount that is within less than 10% of, within less than 5% of, within less than 1% of, within less than 0.1% of, and within less than 0.01% of the stated amount. For example, “about” may mean within 1 or more than 1 standard deviation, per the practice in the art. Alternatively, “about” may mean a range of up to 20%, up to 10%, up to 5%, or up to 1% of a given value. As used herein, the term “about” a number refers to that number plus or minus 10% of that number. The term “about” a range refers to that range minus 10% of its lowest value and plus 10% of its greatest value. Where particular values are described in the application and claims, unless otherwise stated the term “about” meaning within an acceptable error range for the particular value may be assumed.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

The following examples are included for illustrative purposes only and are not intended to limit the scope of the invention.

### Example 1: Orthopedic Surgery—Tendon Laceration & Repair

In this example, the devices and multi-component fluid described herein are used to produce an adhesion barrier, prevent the formation of adhesions between the repaired tendon and surrounding tissue, and to improve patient outcomes following a tendon laceration and repair.

A subject has suffered a laceration, cut, or rupture of the tendon in their hand, a common incident such as when cutting an avocado or bagel while holding it in one's palm. The surgeon is tasked with repairing the lacerated tendon and minimizing the trauma to the subject resulting from surgery, such as preventing the formation of adhesions which can impair the range of motion and require painful physical therapy regimens to restore motion.

The device comprises a 0.6 mm orthopedic applicator and an ECM fluid contained within a syringe comprising a piston and delivered through an annular area of the nozzle. The 0.6 mm orthopedic applicator is connected to a mixer which mixes the ECM fluid to a high viscosity ECM hydrogel as it is dispersed from the nozzle.

The tendon repair is performed, following suturing of the two ends of the tendon, the surgeon places the orthopedic applicator approximately 0.1-2.0 inches away from the suture site and presses the thumb grip, leading to mixing of the multi-component fluid before it is dispensed from the nozzle and delivered as a gel. The surgeon moves the nozzle approximately 180 degrees in each direction relative to a starting point to evenly coat the entire surface of the repaired tendon. The resulting ECM gel has a yield stress between 200 Pa and 700 Pa and evenly coats the surface of the repaired intestinal tissue. The incisions are then closed. The storage modulus of the ECM gel is less than the elastic modulus of the tendons and the surrounding tissue (e.g., 20-40 kPa). The resulting ECM gel forms a layer around the repaired tendon, serving as an adhesion barrier without hindering the biomechanics of tendon function and finger mobility.

Following the tendon repair surgery, once the tendon begins to heal, the formation of scars attaching the tendon to the surrounding sheath is noted to be significantly less due to the ECM gel-based adhesion barrier. As a result, the subject experiences a smoother recovery and restoration of range of motion is achieved through less painful physical therapy regimens, improving patient outcomes significantly.

In the case of a re-injury or if a further surgical procedure is required, due to the prior application of the adhesion barrier, fewer adhesions are noted to have formed between the tendon and surrounding tissues. Consequently, the surgeon expends less time lysing adhesions, thereby reducing risks involved with the subsequent surgical procedure. In addition, the patient undergoes less anesthesia due to reduced surgery length, and overall recovery period is shortened, leading to an improved patient experience.

### Example 2: Abdominal Surgery—Colorectal Resection & Ostomy Creation

In this example, the devices described herein and multi-component fluid of one or more embodiments are used to produce an adhesion barrier, prevent the formation of adhesions along the small intestine, large intestine, and to improve patient outcomes in a colorectal resection, and an ileostomy creation procedure.

A subject has suffered a perforation of the sigmoid colon approximately 8 inches above the rectum. A surgeon is performing colorectal resection of a subject's sigmoid colon a result of the colorectal perforation and diverting the subject's bowel just below the small intestine as part of an ileostomy creation. The surgeon is performing the surgery using a laparoscopic surgery method to minimize the trauma to the subject resulting from the surgery.

The device comprises a 0.6 mm laparoscopic nozzle and an ECM fluid contained within a syringe comprising a piston and delivered through an annular area of the nozzle. The 0.6 mm laparoscopic nozzle is connected to a mixer which mixes the ECM fluid to a high viscosity ECM hydrogel as it is dispersed from the nozzle.

The colorectal recession is performed, following suture of the two sections of healthy colon tissue to one another, the surgeon places a laparoscopic nozzle approximately 0.1-2.0 inches away from the suture site and presses the thumb grip, resulting in mixing of the multi-component fluid before it is dispensed from the nozzle and delivered as a gel. The surgeon moves the nozzle approximately 180 degrees in each direction relative to a starting point to evenly coat the entire surface of the repaired bowel tissue. The resulting ECM gel has a yield stress between 200 Pa and 700 Pa and evenly coats the surface of the repaired intestinal tissue. The laparoscopic incisions are then closed. The storage modulus of the ECM gel is less than the elastic modulus of the sigmoid colon and the surrounding tissue (e.g., 20-40 kPa), and does not impede the biomechanics of bowel function, including bowel movements. The resulting ECM barrier formed over the repaired bowel tissue operates to permit gliding of the abdominal wall over the sigmoid colon tissue and permits gliding of the large intestine tissue over other sections of sigmoid colon tissue it is in contact; and prevents the formation of post-surgical adhesions. Also, as a result of the application of the application of the adhesion barrier, there is a 50% reduction in the formation of scar tissue resulting from the colorectal rescission.

In parallel with the colorectal resection, an ileostomy creation procedure is performed. In this example, an ileostomy procedure is performed by bringing the incised section of the small intestine, the stoma, to the wall abdominal wall and suturing the small intestine in place. At this time, an adhesion barrier is applied to the surface of the incised small intestine tissue, along the sutured section, along the section of tissue extending outward from the body, and along the surface of the tissue remaining within the body, effectively coating the entire section of incised tissue with the ECM barrier.

The surgeon places a dispensing nozzle approximately 0.5-3.0 inches away from the suture site and presses the thumb grip, resulting in mixing of the multi-component fluid before it is dispensed from the nozzle and delivered as a gel. The surgeon moves the nozzle approximately 180 degrees in each direction relative to a starting point to evenly coat the entire surface of the incised and sutured small intestinal tissue. The incisions are then closed with the stoma extending outward from the subject, and an ileostomy bag placed over the stoma. Any remaining CO2 is dissolved in the blood and safely eliminated through normal physiological processes. The storage modulus of the ECM gel is less than the elastic modulus of the large intestine and the surrounding tissue, and does not impede the biomechanics of digestion, or bowel movement.

In the months following the surgery, once the section of the large intestine which has been repaired has begun to heal, when inflammation has receded, and once any resulting infections have been controlled or eradicated, the subject reports for an ileostomy repair procedure.

The ileostomy repair procedure is performed. When the surgeon begins to lyse adhesions, approximately 80% fewer adhesions are noted to have been formed as a result of ileostomy creation, and the adhesions which have been formed are 50% smaller in size relative the size of intestinal adhesions traditionally resulting from such procedures. The ileostomy repair is accomplished in a shorter time as a result of the reduction in time from not lysing a significant number of adhesions, or adhesions of significant size, and the patient is treated with 30% less anesthesia due to the reduced length of the surgery. In addition, the overall risk to the patient resulting from the surgery is significantly reduced due to the reduced number and size of adhesions which need to be lysed, as the lysing of adhesions is traditionally risky due to inhibition of surgeon visibility which raises the risk that a nerve or vessel may be unintentionally cut in the lysing process.

### Example 3: Abdominal Surgery—Colorectal Resection & Ostomy Creation

In this example, the devices described herein and multi-component fluid of one or more embodiments are used to produce an adhesion barrier, prevent the formation of adhesions along the small intestine, large intestine, and to improve patient outcomes in a colorectal resection, and an ileostomy creation procedure.

A subject is undergoing a colorectal resection as a treatment for colon cancer. A surgeon is performing colorectal resection to remove the malignant tissue and is diverting the subject's bowel in the large intestine as part of a colostomy creation. The surgeon is performing the surgery using a laparoscopic surgery method to minimize the trauma to the subject resulting from the surgery.

The device comprises a 0.6 mm laparoscopic nozzle and an ECM fluid contained within a syringe comprising a piston and delivered through an annular area of the nozzle. The 0.6 mm laparoscopic nozzle is connected to a mixer which mixes the ECM fluid to a high viscosity ECM hydrogel as it is dispersed from the nozzle.

The colorectal recession is performed. Following suture of the two sections of healthy colon tissue to one another, the surgeon places a laparoscopic nozzle approximately 0.1-2.0 inches away from the suture site and presses the thumb grip, resulting in mixing of the multi-component fluid before it is dispensed from the nozzle and delivered as a gel. The surgeon moves the nozzle approximately 180 degrees around the intestine in each direction relative to a starting point to evenly coat the entire surface of the repaired bowel tissue, and the surrounding tissues. The resulting ECM gel evenly coats the surface of the repaired intestinal tissue. The laparoscopic incisions are then closed. The storage modulus of the ECM gel is less than the elastic modulus of the large intestine and the surrounding tissue, and does not impede the biomechanics of bowel function, including bowel movements. The resulting ECM barrier formed over the repaired bowel tissue operates to permit gliding of the abdominal wall over the large intestine tissue and permits gliding of the large intestine tissue over other sections of large intestine tissue it is in contact; and prevents the formation of post-surgical adhesions. Also, as a result of the application of the application of the adhesion barrier, there is a 50% reduction in the formation of scar tissue resulting from the colorectal rescission.

In parallel with the colorectal resection, a colostomy creation procedure is performed. In this example, a colostomy procedure is performed by bringing the incised section of the large intestine, the stoma, to the wall abdominal wall and suturing the large intestine in place. At this time, an adhesion barrier is applied to the surface of the incised large intestine tissue, along the sutured section, along the section of tissue extending outward from the body, and along the surface of the tissue remaining within the body, effectively coating the entire section of incised tissue with the ECM barrier.

The surgeon places a laparoscopic nozzle approximately 0.1-2.0 inches away from the suture site and presses the thumb grip, resulting in mixing of the multi-component fluid before it is dispensed from the nozzle and delivered as a gel. The surgeon moves the nozzle approximately 180 degrees in each direction relative to a starting point to evenly coat the entire surface of the incised and sutured small intestinal tissue. The resulting ECM evenly coats the surface of the repaired intestinal tissue. The incisions are then closed with the stoma extending outward from the subject, and a colostomy bag is placed over the stoma. The storage modulus of the ECM gel is less than the elastic modulus of the large intestine and the surrounding tissue, and does not impede the biomechanics of digestion, or bowel movement.

In the months following the surgery, once the section of the large intestine which has been repaired has begun to heal, when inflammation has receded, and once any resulting infections have been controlled or eradicated, the subject report for a colostomy repair procedure.

The colostomy repair procedure is performed. When the surgeon reaches the phase of the repair procedure when it is time lyse adhesions, approximately 80% fewer adhesions are noted to have been formed as a result of colostomy creation, and the few adhesions which have been formed are 50% smaller in size relative the size of intestinal adhesions traditionally resulting from such procedures. The colostomy repair is accomplished in a shorter time as a result of the reduction in time from not lysing a significant number of adhesions, or adhesions of significant size, and the patient is treated with 30% less anesthesia due to the reduced length of the surgery. In addition, the overall risk to the patient resulting from the surgery is significantly reduced due to the reduced number and size of adhesions which need to be lysed, as the lysing of adhesions is traditionally risky due to inhibition of surgeon visibility which raises the risk that a nerve or vessel may be unintentionally cut in the process.

### Example 4: Pelvic Surgery—Cesarean Section

In this example, the devices described herein and multi-component fluid of one or more embodiments are used to produce an adhesion barrier, prevent the formation of adhesions on the fallopian tubes and uterus, as to improve patient outcomes in a caesarian section.

The device comprises a 0.6 mm laparoscopic nozzle and an ECM fluid contained within a syringe comprising a piston and delivered through an annular area of the nozzle. The 0.6 mm laparoscopic nozzle is connected to a mixer which mixes the ECM fluid to a high viscosity ECM hydrogel as it is dispersed from the nozzle.

A subject is undergoing a caesarian section following an extended delivery and upon deceleration of neonate heart rate. A caesarian section is performed, inadvertently resulting in a partial rupture of a fallopian tube. Following removal of the neonate and prior closing of the uterine wall, the surgeon places a dispensing nozzle approximately 0.5-3.0 inches away from the fallopian tubes and presses the thumb grip, resulting in mixing of the multi-component fluid before it is dispensed from the nozzle and delivered as a gel. The surgeon moves the nozzle approximately 180 degrees in each direction relative to a starting point to evenly coat the interior of the uterus and the fallopian tube with the adhesion barrier. The resulting ECM gel evenly coats the surface of the repaired uterine tissue. The storage modulus of the ECM gel is less than the elastic modulus of the uterine wall, fallopian tubes, and the surrounding tissue, and does not impede the biomechanics of ovulation, and menstruation. The surgeon then proceeds with manual closure of the uterine wall via sutures or other methods.

Following closure of the uterine wall via suture, the surgeon places a dispensing nozzle approximately 3.0-5.0 inches away from the uterine wall and presses the thumb grip, resulting in mixing of the multi-component fluid before it is dispensed from the nozzle and delivered as a gel. The surgeon moves the nozzle approximately 130 degrees in an arc across the uterine wall of the subject to evenly coat the surface of the uterine wall with the adhesion barrier. The resulting ECM gel evenly coats the surface of the repaired uterine wall. The storage modulus of the ECM gel is less than the elastic modulus of the uterine wall the surrounding tissue, and does not impede the biomechanics of movement of the uterus in the peritoneal cavity.

Following the surgery, the subject experiences a 75% reduction in formation of scar tissue along the fallopian tubes and uterus. As a result of the reduction of scar tissue in the fallopian tubes, the subject is able to continue experiencing normal ovulation as eggs descend from the fallopian tube, lowering the risk of infertility as a result of the C-section. As a result of the reduction in scar tissue along the uterine wall, future embryos are less likely to cause abnormal expansion of tissue at the site of the first C-section, thus reducing the risk of uterine wall rupture. Similarly, the reduction of scar tissue along the fallopian tube reduces the risk of ectopic pregnancy. The subject further experiences a 75% reduction in the formation of adhesions between the uterine wall and the peritoneal activity, and a 50% reduction in adhesion size of adhesions. Overall, the subject experiences improved healing, reduced scarring, reduced adhesions, ongoing fertility, and reduced risk of complications in subsequent pregnancies resulting from the C-section.

### Example 5: Biopsy Collection

In this example, the devices described herein and multi-component fluid of one or more embodiments are used to produce an adhesion barrier and prevent formation of scar tissue at the location of a biopsy, as to improve patient outcomes following the biopsy.

The device comprises a 0.6 mm laparoscopic nozzle and an ECM fluid contained within a syringe comprising a piston and delivered through an annular area of the nozzle. The 0.6 mm laparoscopic nozzle is connected to a mixer which mixes the ECM fluid to a high viscosity ECM hydrogel as it is dispersed from the nozzle.

A subject is undergoing biopsy collection for analysis to determine the cause of an abnormal skin condition under suspicion of malignancy. The biopsy is performed. Following collection of the sample and closure of the incision site with a suture or other means, the surgeon places a dispensing nozzle approximately 3.0-5.0 inches away from the incision site and presses the thumb grip, resulting in mixing of the multi-component fluid before it is dispensed from the nozzle and delivered as a gel. The surgeon evenly coats the incision site with the adhesion barrier. The resulting ECM gel evenly coats the surface of the repaired dermal tissue. Following application of the ECM gel adhesion barrier to the surface of the skin, keloid formation is reduced, resulting in a 25% reduction in scar tissue at the incision site as it recovers, and a normalized rate of melanin production at the incision site. Overall, the patient outcome is improved as result of the reduction in formation of scar tissue at the incision site.

### Example 6: Cardiac Surgery

In this example, the devices described herein and multi-component fluid of one or more embodiments are used to produce an adhesion barrier in the thoracic cavity following installation of a left ventricular assist device (LVAD).

The device comprises a 0.6 mm laparoscopic nozzle and an ECM fluid contained within a syringe comprising a piston and delivered through an annular area of the nozzle. The 0.6 mm laparoscopic nozzle is connected to a mixer which mixes the ECM fluid to a high viscosity ECM hydrogel as it is dispersed from the nozzle.

The subject is prepared for installation of a LVAD, and installation of the device is performed. Prior to closure the thoracic cavity, the surgeon places a dispensing nozzle approximately 1.0-3.0 inches away from the cardiac tissue and presses the thumb grip, resulting in mixing of the multi-component fluid before it is dispensed from the nozzle and delivered as a gel. The surgeon moves the nozzle in a 130 degree arc relative to a starting point to evenly coat the interior of the thoracic cavity with the adhesion barrier. The resulting ECM gel evenly coats the surface of the thoracic cavity. The storage modulus of the ECM gel is less than the elastic modulus of the heart muscles, arteries, and veins, and other the surrounding tissue, and does not impede the biomechanics of ventricular contractions and blood flow through the left ventricle. The surgeon may then proceed with closure of the thoracic cavity.

Following the installation of the LVAD, the subject experiences a reduction in formation of scar tissue, reduced formation of adhesions in the thoracic cavity, increased cardiovascular capacity, and an improved patient outcome.

Following installation of the LVAD, the subject later requires a heart transplant. The subject is prepared for a heart transplant. Prior to transplant of the donor heart into the subject, the surgeon must prepare the thoracic cavity for the donor heart by removing the subject's failing heart. Due to application of the ECM gel-based adhesion barrier, there is a significant reduction formation of scar tissue surrounding the LVAD. The surgeon removes approximately 50% less scar tissue surrounding the LVAD in order to access the heart than generally would have been present in the absence of application of the adhesion barrier. As a result of the significant reduction in scar tissue development, the surgeon is required to expend significantly less time in removing the scar tissue while the donor heart remains on ice. Similarly, there is an 85% reduction in formation of adhesions in the thoracic cavity resulting from the prior surgery, and the surgeon is required to expend significantly less time lysing adhesions when removing the subject's failing heart. Due to the donor heart remaining on ice for shortened period while the scar tissue is removed and adhesions are lysed, there is remarkably reduced risk of the donor heart failing to regain full function, resulting in a decreased risk of patient death.

### Example 7: Rheology Testing of Yield Stress of Devices Described Herein

A study was conducted to determine if yield stress measurements can be taken without tissue sections by comparing hydrogel only samples to hydrogels adhered to tissue and to determine a target threshold for yield stress of a hydrogel or multi-component fluid as described herein.

The multi-component fluid was made using a digest dialyzed against 17.5 mM acetic acid and a neutralization buffer.

Four types of dispensing methods were compared: 1) a 3 mL syringe with a standard nozzle, 2) the device of FIGS. 4A and 4B, 3) the device of FIG. 1A (“airless”), and 4) the device of FIG. 3 (“gas”).

For the first method, the digest and buffer were manually mixed using standard methods, wherein 0.5 mL of digest and 0.5 mL of neutralization buffer were each collected in separate 3 mL syringes. The 2 syringes were connected tip to tip with a Luer-Luer connector, carefully removing the air in the connector. The buffer and digest were mixed back and forth 25 times until there was a homogenous pre-gel mixture. The nozzle had 2 Luer ports. The pre-gel was injected into 1 port, and compressed air was injected into the other port. The compressed air and pre-gel mixed at the tip to disperse the pre-gel in small droplets. The air pressure at which the air passed through the nozzle was set between 5-10 psi to ensure aerosolization of the hydrogel without removing (blowing away) the hydrogel already dispensed to the application site.

For the second method, the applicator was designed to receive a pair prefilled 3 mL syringes (1 containing ECM digest, 1 containing buffer). Actuation of plungers dispensed the hydrogel through a mixer onto the application site. Furthermore, the applicator contained a small CO2 cylinder for gas-assisted dispersing, but gas-assisted dispersing was not used in this study.

For the third and fourth method, both the airless and gas-assisted applicators were designed to receive a pair prefilled 3 mL glass cartridges (1 containing ECM digest, 1 containing buffer). Actuation of 3D printed plunger rods dispensed the hydrogel through a mixer onto the application site. In the case of the gas-assisted applicator, an additional Luer port was present to receive dispersant gas from an airline.

Rheology was done to test the adhesive strength, as indicated by yield stress, of the hydrogel when a certain dispensing method was used. Porcine intestine was used as the tissue substrate for yield stress testing on the rheometer. The tissue was rinsed twice with water, cut open and laid flat with the intralumenal surface facing up. A scraper was used to remove all the layers of the intestinal wall, leaving only behind the serosa. The intestine was then super-glued to a transparency paper which allowed the intestine to be secured to the rheometer base with double sided tape, limiting its movement during the procedure. A 40 mm stainless steel plate was used for testing. The hydrogel was dispensed using a syringe pump in the center of the rheometer base and a time sweep procedure was run to measure the peak storage modulus. Immediately after, a flow ramp where the amount of stress on the hydrogel increased at a constant rate was run to determine the hydrogel yield stress.

A rheology protocol evaluating both gelation kinetics (e.g., change in modulus) and yield stress was run in 10 replicates since tissue samples can have greater variance. The geometry setting used for tests without tissue was a 0.25 mm gap between the 40 mm plate and the rheometer base. For testing done with tissue, the geometry gap was set to 0.5 mm and the volume of hydrogel dispensed was double to ensure the hydrogel was being measured, rather than the tissue.

The first step of the procedure was a “Time Sweep” for a total of 5 minutes, run at 37° C. with a strain of 1%. This test was used to determine gelation rate and final hydrogel storage modulus.

The second step was a “Flow ramp” that increases the stress applied to the hydrogel at a constant rate from 1 Pa to 5000 Pa. This test was used to determine the tissue adhesive strength of the hydrogel determined by the amount of stress applied at the peak hydrogel viscosity.

### Results

TABLE 1
Peak Storage Modulus, Time to 300 Pa, and Yield Stress of Different Devices

| | Peak Storage Modulus | | Time to 300 Pa | | Yield Stress | |
|---|---|---|---|---|---|---|
| | (Pa) | | (s) | | (Pa) | |
| | (mean ± SEM) | | (mean ± SEM) | | (mean ± SEM) | |
| Dispensing Method | Tissue | No tissue | Tissue | No tissue | Tissue | No tissue |
| Standard Nozzle | 1143 ± 63 | 477 ± 9 | 138 ± 5 | 76 ± 2 | 202 ± 8 | 241 ± 39 |
| Applicator - 2022 | 1096 ± 52 | 467 ± 17 | 132 ± 4 | 75 ± 2 | 162 ± 8 | 105 ± 13 |
| Airless Applicator | 1280 ± 81 | 565 ± 8 | 156 ± 6 | 63 ± 1 | 204 ± 18 | 371 ± 49 |
| Gas-assisted | 883 ± 34 | 501 ± 23 | 140 ± 5 | 67 ± 1 | 228 ± 11 | 693 ± 46 |
| Applicator | | | | | | |

The modulus and time to 300 Pa was different in the presence and absence of tissue.

The key measurement reported is the yield stress. As seen in FIGS. 9A and 9B, the trend in performance across the set of application methods was the same in the presence or absence of tissue. For example, the weakest yield stress was produced by the second method using the device of FIGS. 4A and 4B (labeled ‘Turkey’ in FIGS. 9A and 9B) with or without tissue. The best performer (the gas-assisted device of FIG. 3) was also consistent with and without tissue.

The other finding was that the device of FIGS. 4A and 4B, which produced positive sub-optimal results in vivo, was the only applicator to produce a yield stress below 200 Pa. All other applicator produced more positive results in vivo.

While preferred embodiments of the present disclosure have been shown and described herein, it will be obvious to those skilled in the art that such embodiments are provided by way of example only. Numerous variations, changes, and substitutions will now occur to those skilled in the art without departing from the scope of the present disclosure. It should be understood that various alternatives to the embodiments of the present disclosure described herein may be employed in practicing the invention. It is intended that the following claims define the scope of the invention and that methods and structures within the scope of these claims and their equivalents be covered thereby.

1. A device for delivery of a multi-component fluid, the device comprising:
a housing having a first lumen and a second lumen, wherein the first lumen is configured to receive a first component of the multi-component fluid, and the second lumen is configured to receive a second component of the multi-component fluid;
a mixer comprising one or more mixer stages and one or more vortex inducers, wherein a proximal end of the mixer is in fluid communication with the first and second lumens, wherein the proximal end is configured to receive the first component and the second component within the mixer and to mix the first component and the second component to form the multi-component fluid; and
a nozzle coupled to the mixer, wherein the nozzle comprises a nozzle outlet configured to receive the multi-component fluid from the mixer and deliver the multi-component fluid from the nozzle outlet.

2. The device of claim 1, wherein the nozzle further comprises one or more spacers.

3. The device of claim 2, wherein at least one vortex inducer of the one or more vortex inducers is located distal to the one or more spacers and proximal to the mixer.

4. The device of claim 1, wherein one or more vortex inducers are located proximal to the one or more spacers.

5. The device of claim 1, wherein the one or more spacers are proximal to at least one vortex inducer of the one or more vortex inducers, and wherein the one or more spacers are distal to at least a second vortex inducer.

6. The device of claim 2, wherein a distal end of the spacer is narrower than a proximal end of the spacer.

7. The device of claim 1, wherein the nozzle comprises two vortex inducers.

8. The device of claim 1, wherein a vortex inducer of the one or more vortex inducers comprises the nozzle outlet.

9. The device of claim 1, wherein the one or more vortex inducers comprise high shear mixers of the multi-component fluid.

10. The device of claim 1, wherein the mixer comprises twelve mixer stages.

11. The device of claim 10, wherein the twelve mixer stages are configured to form an array of mixer stages.

12. The device of claim 11, wherein the array of mixer stages further comprises the one or more vortex inducers, or the one or more spacers, or both.

13. The device of claim 1, wherein the first component or the second component comprises a buffer.

14. The device of claim 13, wherein the buffer solution is a phosphate buffered saline.

15. The device of claim 1, wherein the multi-component fluid is buffered to a mildly acidic pH.

16. The device of claim 15, wherein the multi-component fluid is buffered to a pH from about 6.5 to about 8.0.

17. The device of claim 1, wherein the first component or the second component comprises an extracellular (ECM) matrix material.

18. The device of claim 1, wherein the mixer is a high shear mixer.

19. The device of claim 1, wherein the vortex inducer is configured to induce a rotational component in the multi-component fluid flowing therethrough.

20. The device of claim 1, wherein the vortex inducer imparts shear stress on the multi-component fluid flowing therethrough.

21. The device of claim 20, wherein the shear stress imparted on the multi-component fluid results in a gel comprising a yield stress of at least 200 Pa.

22. The device of claim 20, wherein the shear stress imparted on the multi-component fluid results in a gel comprising a yield stress of between about 200 Pa to about 700 Pa.

23. The device of claim 1, wherein the multi-component fluid comprises a hydrogel.

24. The device of claim 1, wherein the mixer comprises a baffle, a blade, a channel, a semi-circular cylinder, a slot, a plate, a fin, or a combination thereof.

25. A method of delivering a multi-component fluid for treatment of a wound, the method comprising:
a) delivering a first component to a first lumen and a second component to a second lumen of a delivery device, wherein the delivery device comprises:
(i) a housing having a first lumen and a second lumen, wherein the first lumen is configured to receive a first component of the multi-component fluid, and the second lumen is configured to receive a second component of the multi-component fluid;
(ii) a mixer comprising one or more mixer stages and one or more vortex inducers, wherein a proximal end of the mixer is in fluid communication with the first and second lumens, wherein the proximal end is configured to receive the first component and the second component within the mixer and to mix the first component and the second component to form the multi-component fluid;
(iii) a nozzle coupled to the mixer, wherein the nozzle comprises a nozzle outlet configured to receive the multi-component fluid from the mixer and deliver the multi-component fluid from the nozzle outlet; and
(iv) an actuator coupled to the housing;
b) activating the actuator so that the mixer receives the first component and the second component;
c) further activating the actuator so that the first component and the second component mix to form the multi-component fluid; and
d) further activating the actuator so that the multi-component fluid is delivered from the nozzle outlet onto a patient's wound.

26. The method of claim 25, wherein the first lumen of the delivery device is in fluid communication with a first container, such that the first lumen is configured to receive the first component of the multi-component fluid from the first container.

27. The method of claim 25, wherein the second lumen of the delivery device is in fluid communication with a second container, such that the second lumen is configured to receive the second component of the multi-component fluid from the second container.

28. The method of claim 25, wherein the actuator of the delivery device comprises a piston, a plunger, a pump, or a multi-stage button.

29. (canceled)

30. A method for delivering a multi-component fluid, the method comprising:
a) delivering a first component and a second component of the multi-component fluid through a first lumen and a second lumen, respectively, of a delivery device, wherein the delivery device comprises:
(i) a housing having a first lumen and a second lumen, wherein the first lumen is configured to receive a first component of the multi-component fluid, and the second lumen is configured to receive a second component of the multi-component fluid;
(ii) a mixer comprising one or more mixer stages and one or more vortex inducers, wherein a proximal end of the mixer is in fluid communication with the first and second lumens, wherein the proximal end is configured to receive the first component and the second component within the mixer and to mix the first component and the second component to form the multi-component fluid; and
(iii) a nozzle coupled to the mixer, wherein the nozzle comprises a nozzle outlet configured to receive the multi-component fluid from the mixer and deliver the multi-component fluid from the nozzle outlet;
b) delivering the multi-component fluid through one or more vortex inducers in the mixer; and
c) delivering the multi-component fluid through the nozzle outlet.

31. The method of claim 30, wherein an actuator is coupled to the housing, and the method further comprises activating the actuator so that the mixer receives the first component and the second component.

32. (canceled)

33. (canceled)

34. The method of claim 25, further comprising delivering the first component and the second component to the mixer, wherein delivering the first component and the second component to the mixer includes moving the first component and the second component from the first lumen and the second lumen, respectively, to a third lumen comprising both the first component and the second component.

35. The method of claim 34, further comprising imparting shear stress onto the multi-component fluid in the third lumen.
